(19) United States
(12) Patent Application Publication
(10) Pub. No.: US 2006/0292081 A1
(43) Pub. Date: Dec. 28, 2006

(54) Methods for preparing pharmaceutical compositions

(75) Inventors: David Morton, Wiltshire (GB); Yorick Kamlag, Wiltshire (GB)

Correspondence Address: DAVIDSON, DAVIDSON & KAPPEL, LLC, 485 SEVENTH AVENUE, 14TH FLOOR, NEW YORK, NY, 10018, US

(73) Assignee: Vectura Limited, Chippenham (GB)

(21) Appl. No.: 10/570,902

(22) PCT Filed: Sep. 15, 2004
(86) PCT No.: PCT/GB04/03938

§ 371 Date: Jun. 19, 2006

(30) Foreign Application Priority Data
Sep. 15, 2003 (GB) 0321608.2
Apr. 23, 2004 (GB) 0409133.6

(51) Int. Cl. A61K 9/14; A61M 11/00; A61L 9/04

(52) U.S. Cl. 424/46; 128/200.23

(57) ABSTRACT

The present invention relates to improvements in dry powder formulations comprising a pharmaceutically active agent for administration by inhalation, and in particular to methods of preparing dry powder compositions with improved properties. In particular, spray drying processes are adapted and adjusted to obtain active particles with higher fine particle fractions and fine particle doses.

The present invention relates to improvements in dry powder formulations comprising a pharmaceutically active agent for administration by inhalation, and in particular to methods of preparing dry powder compositions with improved properties.

The lung provides an obvious target for local administration of formulations which are intended to cure or alleviate respiratory or pulmonary diseases, such as cystic fibrosis (CF), asthma, lung cancer, etc. The lung also provides a route for delivery of systemically acting formulations to the blood stream, for example, for delivery of active agents which are not suitable for oral ingestion, such as agents that degrade in the digestive tract before they can be absorbed, and those requiring an extremely rapid onset of their therapeutic action.

It is well established that delivering pharmaceutically active agents to the lung by pulmonary inhalation of a dry powder has a number of advantages which make this an attractive mode of delivery.

However, the delivery of dry powder particles of pharmaceutical products to the respiratory tract presents certain problems. The inhaler device, which is preferably a bespoke device, such as a dry powder inhaler (DPI), should deliver the maximum possible proportion of the particles of pharmaceutically active agent (active particles) to the lungs. Indeed, a significant proportion of the active particles should be deposited in the lower lung, preferably even at the low inhalation capabilities to which some patients, especially asthmatics, are limited. However, when using many dry powder formulations, it has been found that frequently only a small proportion (often only about 10%) of the active particles that leave the device on actuation are actually deposited in the lower lung. As a result, much work has been done on improving dry powder formulations to enhance the delivery of the active particles to the lower respiratory tract or deep lung.

The type of dry powder inhaler used can influence the proportion of the active particles delivered to the lung, as different types of inhaler devices provide different air flow conditions which lead to the active particles reaching the respiratory tract. Also, the physical properties of the powder affect both the efficiency and reproducibility of delivery of the active particles and the site of deposition in the respiratory tract.

On exit from the inhaler device, the active particles should form a physically and chemically stable aerocolloid which remains in suspension until it reaches a conducting bronchiole or smaller branching of the pulmonary tree or other absorption site, preferably in the lower lung. Once at the absorption site, the active particles should be capable of efficient collection by the pulmonary mucosa with as few as possible active particles being exhaled from the absorption site.

When delivering a formulation to the lung for local or systemic action, the size of the active particles within the formulation is very important in determining the site of the absorption in the body.

For formulations to reach the deep lung or the blood stream via inhalation, the active agent in the formulation must be in the form of very fine particles, for example, having a mass median aerodynamic diameter (MAD) of less than 10 μm.

It is well established that particles having an MMAD of greater than 10 on are likely to impact on the walls of the throat and generally do not reach the lung. Particles having an MMAD in the region of 5 to 2 μm will generally be deposited in the respiratory bronchioles whereas particles having an MMAD in the range of 3 to 0.05 μm are likely to be deposited in the alveoli and to be absorbed into the bloodstream.

Preferably, for delivery to the lower respiratory tract or deep lung, the MMAD of the active particles is not more than 10 μm, and preferably not more than 5 μm, more preferably not more than 3 μm, and may be less than 2 μm, less than 1.5 μm or less than 1 μm. Ideally, at least 90% by weight of the active particles in a dry powder formulation should have an aerodynamic diameter of not more than 10 μm, preferably not more than 5 μm, more preferably not more than 3 μm, not more than 2.5 μm, not more than 2.0 μm, not more than 1.5%, or even not more than 1.0 μm.

When dry powders are produced using conventional processes, the active particles will vary in size, and often this variation can be considerable. This can make it difficult to ensure that a high enough proportion of the active particles are of the appropriate size for administration to the correct site. It is therefore desirable to have a dry powder formulation wherein the size distribution of the active particles is as narrow as possible. For example, the geometric standard deviation of the active particle aerodynamic or volumetric size distribution (σg), is preferably not more than 2, more preferably not more than 1.8, not more than 1.6, not more than 1.5, not more than 1.4, or even not more than 1.2. This will improve dose efficiency and reproducibility.

Fine particles, that is, those with an MMAD of less than 10 μm and smaller, tend to be increasingly thermodynamically unstable as their surface area to volume ratio increases, which provides an increasing surface free energy with this decreasing particle size, and consequently increases the tendency of particles to agglomerate and the strength of the agglomerate. In the inhaler, agglomeration of fine particles and adherence of such particles to the walls of the inhaler are problems that result in the fine particles leaving the inhaler as large, stable agglomerates, or being unable to leave the inhaler and remaining adhered to the interior of the inhaler, or even clogging or blocking the inhaler.

The uncertainty as to the extent of formation of stable agglomerates of the particles between each actuation of the inhaler, and also between different inhalers and different batches of particles, leads to poor dose reproducibility. Furthermore, the formation of agglomerates means that the MMAD of the active particles can be vastly increased, with agglomerates of the active particles not reaching the required part of the lung.

The metered dose (MD) of a dry powder formulation is the total mass of active agent present in the metered form presented by the inhaler device in question. For example, the MD might be the mass of active agent present in a capsule for a Cyclohaler (trademark), or in a foil blister in an Aspirair (trademark) device.

The emitted dose (ED) is the total mass of the active agent emitted from the device following actuation. It does not include the material left on the internal or external surfaces of the device, or in the metering system including, for example, the capsule or blister. The ED is measured by collecting the total emitted mass from the device in an apparatus frequently identified as a dose uniformity sampling apparatus (DUSA), and recovering this by a validated quantitative wet chemical assay (a gravimetric method is possible, but this is less precise).

The fine particle dose (FPD) is the total mass of active agent which is emitted from the device following actuation which is present in an aerodynamic particle size smaller than a defined limit. This limit is generally taken to be 5 μm if not expressly stated to be an alternative limit, such as 3 μm, 2 μm or 1 μm, etc. The FPD is measured using an impactor or impinger, such as a twin stage impinger (TSI), multi-stage impinger (MSI), Andersen Cascade Impactor (ACI) or a Next Generation Impactor (NGI). Each impactor or impinger has a pre-determined aerodynamic particle size collection cut points for each stage. The FPD value is obtained by interpretation of the stage-by-stage active agent recovery quantified by a validated quantitative wet chemical assay (a gravimetric method is possible, but this is less precise) where either a simple stage cut is used to determine FPD or a more complex mathematical interpolation of the stage-by-stage deposition is used.

The fine particle fraction (FPF) is normally defined as the FPD divided by the ED and expressed as a percentage. Herein, the FPF of ED is referred to as FPF(ED) and is calculated as FPF(ED)=(FPD/ED)×100%.

The fine particle fraction (FPF) may also be defined as the FPD divided by the MD and expressed as a percentage. Herein, the FPF of MD is referred to as FPF(MD), and is calculated as FPF(MD)=(FPD/MD)×100%.

The tendency of fine particles to agglomerate means that the FPF of a given dose is often highly unpredictable and a variable proportion of the fine particles will be administered to the lung, or to the correct part of the lung, as a result.

In an attempt to improve this situation and to provide a consistent FPF and FPD, dry powder formulations may include additive material.

The additive material is intended to decrease the adhesion and cohesion experienced by the particles in the dry powder formulation. It is thought that the additive material interferes with the weak bonding forces between the small particles, helping to keep the particles separated and reducing the adhesion of such particles to one another, to other particles in the formulation if present and to the internal surfaces of the inhaler device. Where agglomerates of particles are formed, the addition of particles of additive material decreases the stability of those agglomerates so that they are more likely to break up in the turbulent air stream and collisions created on actuation of the inhaler device, whereupon the particles are expelled from the device and inhaled. As the agglomerates break up, the active particles return to the form of small individual particles which are capable of reaching the lower lung.

In the prior art, dry powder formulations are discussed which include additive material in particulate form, the particles generally being of a size comparable to the size of the fine active particles. In some embodiments, the additive material may form a coating, generally a discontinuous coating, on the active particles and/or any carrier particles.

Preferably, the additive material is an anti-adherent material and it will tend to reduce the cohesion between particles and will also prevent fine particles becoming attached to the inner surfaces of the inhaler device. Advantageously, the additive material is an anti-friction agent or glidant and will give better flow of the pharmaceutical composition in the inhaler. The additive materials used in this way may not necessarily be usually referred to as anti-adherents or anti-friction agents, but they will have the effect of decreasing the adhesion and cohesion between the particles or improving the flow of the powder. The additive materials are often referred to as force control agents (FCAs) and they usually lead to better dose reproducibility and higher fine particle fractions.

Therefore, an FCA, as used herein, is an agent whose presence on the surface of a particle can modify the adhesive and cohesive surface forces experienced by that particle, in the presence of other particles. In general, its function is to reduce both the adhesive and cohesive forces.

In general, the optimum amount of additive material to be included in a dry powder formulation will depend on the chemical composition and other properties of the additive material and of the active material, as well as the nature of other particles such as carrier particles, if present. In general, the efficacy of the additive material is measured in terms of the fine particle fraction of the composition.

Known additive materials usually consist of physiologically acceptable material, although the additive material may not always reach the lung. For example, where the additive particles are attached to the surface of carrier particles, they will generally be deposited, along with those carrier particles, at the back of the throat of the user.

Preferred additive materials used in the prior art dry powder formulations include amino acids, peptides and polypeptides having a molecular weight of between 0.25 and 1000 kDa and derivatives thereof, dipolar ions such as zwitterions, lipids and phospholipids such as lecithin, and metal stearates such as magnesium stearate.

In a further attempt to reduce agglomeration of the fine active particles and to provide a consistent FPF and FPD, dry powder formulations often include coarse carrier particles of excipient material mixed with the fine particles of active material. Rather than sticking to one another, the fine active particles tend to adhere to the surfaces of the coarse carrier particles whilst in the inhaler device, but are supposed to release and become dispersed upon actuation of the dispensing device and inhalation into the respiratory tract, to give a fine suspension. The carrier particles preferably have MMADs greater than 60 μm.

The inclusion of coarse carrier particles is also very attractive where very small doses of active agent are dispensed. It is very difficult to accurately and reproducibly dispense very small quantities of powder and small variations in the amount of powder dispensed will mean large variations in the dose of active agent where the powder comprises mainly active particles. Therefore, the addition of a diluent, in the form of large excipient particles will make dosing more reproducible and accurate.

Carrier particles may be of any acceptable excipient material or combination of materials. For example, the carrier particles may be composed of one or more materials selected from sugar alcohols, polyols and crystalline sugars. Other suitable carriers include inorganic salts such as sodium chloride and calcium carbonate, organic salts such as sodium lactate and other organic compounds such as polysaccharides and oligosaccharides. Advantageously, the carrier particles are composed of a polyol. In particular, the carrier particles may be particles composed of crystalline sugar, for example mannitol, dextrose or lactose. Preferably, the carrier particles are of lactose.

Advantageously, substantially all (by weight) of the carrier particles have a diameter which lies between 20 μm and 1000 μm, more preferably 50 μm and 1000 μm. Preferably, the diameter of substantially all (by weight) of the carrier particles is less than 355 μm and lies between 20 μm and 250 μm.

Preferably, at least 90% by weight of the carrier particles have a diameter between from 30 μm to 180 μm. The relatively large diameter of the carrier particles improves the opportunity for other, smaller particles to become attached to the surfaces of the carrier particles and to provide good flow and entrainment characteristics, as well as improved release of the active particles in the airways to increase deposition of the active particles in the lower lung.

The ratios in which the carrier particles (if present) and composite active particles are mixed will, of course, depend on the type of inhaler device used, the type of active particles used and the required dose. The carrier particles may be present in an amount of at least 50%, more preferably 70%, advantageously 90% and most preferably 95% based on the combined weight of the composite active particles and the carrier particles.

However, a further difficulty is encountered when adding coarse carrier particles to a composition of fine active particles and that difficulty is ensuring that the fine particles detach from the surface of the large particles upon actuation of the delivery device.

The step of dispersing the active particles from other active particles and from carrier particles, if present, to form an aerosol of fine active particles for inhalation is significant in determining the proportion of the dose of active material which reaches the desired site of absorption in the lungs. In order to improve the efficiency of that dispersal, it is known to include in the composition additive materials, including FCAs of the nature discussed above. Compositions comprising fine active particles and additive materials are disclosed in WO 97/03649 and WO 96/23485.

In light of the foregoing problems associated with known dry powder formulations, even when they include additive material and/or carrier particles, it is an aim of the present invention to provide dry powder compositions which have physical and chemical properties which lead to an enhanced FPF and FPD. This will lead to greater dosing efficiency, with a greater proportion of the dispensed active agent reaching the desired part of the lung for achieving the required therapeutic effect.

In particular, the present invention seeks to optimise the preparation of particles of active agent used in the dry powder composition by engineering the particles making up the dry powder composition and, in particular, by engineering the particles of active agent. It is an aim of the present invention to provide particles of active agent which are very small and therefore suitable for pulmonary inhalation.

These particles may be smaller than those produced by known methods or processes. It is also an aim to provide particles with a particle make-up and morphology which will produce high FPF and FPD results, even when the particles are very small.

Thus, it is an aim of the present invention to provide fine particles which have a reduced tendency to agglomerate or to form hard agglomerates, that is, agglomerates which are not substantially broken up when the powder is dispensed from an inhaler device. It is further important that the particles are made by a method or process which is relatively cheap and simple.

Whilst the FPF and FPD of a dry powder formulation are dependent on the nature of the powder itself, these values are also influenced by the type of inhaler used to dispense the powder. Dry powder inhalers can be “passive” devices in which the patient's breath is the only source of gas which provides a motive force in the device. Examples of “passive” dry powder inhaler devices include the Rotahaler and Diskhaler (GlaxoSmithKline) and the Turbohalet (Astra-Draco) and Novolizer (trade mark) (Viatris GmbH). Alternatively, “active” devices may be used, in which a source of compressed gas or alternative energy source is used. Examples of suitable active devices include Aspirair (trade mark) (Vectura Ltd—see WO 01/00262 and GB2353222) and the active inhaler device produced by Nektar Therapeutics (as covered by U.S. Pat. No. 6,257,233). As a rule, the FPF obtained using a passive device will tend not to be as good as that obtained with the same powder but using an active device.

It is another aim of the present invention to optimise the powder properties, so that the FPF and FPD are improved compared to those obtained using known powder formulations, regardless of the type of device used to dispense the composition of the invention.

In the past, several methods have been used to make fine particles of active material. The material may be ground or milled to form particles with the desired size. Alternatively, the particles may be made by spray drying techniques. Some other alternative methods include various forms of supercritical fluid processing, spray-freeze drying, and various forms of precipitation and crystallisation from bulk solution.

The present invention is concerned with improving the conventional spray drying techniques, in order to produce active particles with enhanced chemical and physical properties so that they perform better when dispensed from a DPI than particles formed using conventional spray drying techniques, providing a greater FPF and FPD for any given dispensing device. The improved results are preferably achieved regardless of whether the DPI used to dispense the powder is an active inhaler or a passive inhaler.

Spray drying is a well-known and widely used technique for producing particles of material. To briefly summarise, the material to be made into particles is dissolved or dispersed in a liquid, or can be made into a liquid. This liquid is then sprayed through a nozzle under pressure to produce a mist or stream of fine liquid droplets. These fine droplets are usually exposed to heat which rapidly evaporates the excess volatile liquid in the droplets, leaving effectively dry powder particles. The process is relatively cheap and simple.

A standard method for producing particles of an active material involves using a conventional spray dryer, such as a Büchi B-191 under a “standard” set of parameters. Such standard parameters are set out in Table 1. Alternative conventional spray driers are widely available from several other companies including Niro and Lab Plant.

TABLE 1
“Standard” parameters used in spray drying using the Büchi
B-191 spray dryer (Büchi two-fluid nozzle, internal setting,
0.7 mm mixing needle and cap, 100% aspirator setting)

| | | | Total solid | | |
|---|---|---|---|---|---|
| | | | conc'n | Solvent | Feed |
| Atomisation | Inlet | Outlet | (% w/w) | (host | rate |
| pressure | temp | temp | in solvent | liquid) | (ml/min) |
| 5-6 bar | 150° C. | ˜100° C. | 1 | Aqueous | 5 |

There are a number of problems associated with the spray drying of pharmaceutically active agents. Firstly, there is the problem that the conventional spray drying processes and apparatus have a relatively low output for very fine powders (e.g. <10 μm) and therefore are not particularly well suited to large scale production of pharmaceuticals. Secondly, most spray drying involves exposing the spray dried material to high temperatures, in order to ensure that the necessary evaporation takes place so that the dry particles are formed. Some temperature-sensitive active agents can be adversely affected by exposure to the temperatures used in conventional spray drying methods. The high temperatures used for drying (for example 80 to 250° C.) can lead to very rapid evaporation, and often the drying rate of droplets is uneven. This can lead to undesirable particle features, such as a range of different particle structure and morphologies. A further disadvantage associated with conventional spray drying techniques is that the particles produced can have a broad range of particle sizes. This is due to the nature of the conventional spray nozzles, such as the two-fluid nozzles, that produce the droplets of liquid. The range of particle sizes means that whilst some of the particles produced have the desired particle size, a proportion of the particles will not. Furthermore, this often results in a considerable quantity of the material, by mass, being larger than the desired particles size for delivery to the required site in the lung. A further disadvantage associated with conventional spray drying techniques is that the droplets generally tend to be produced with very high velocities, and this can produce undesirable features as outlined below.

Despite the foregoing problems, spray drying pharmaceutically active agents is still an accepted method of producing particles which are of a size suitable for administration by dry powder inhalation to the lungs.

Whilst spray drying can produce particles of a small enough size to be inhaled into the deep lung, these particles will frequently suffer from the agglomeration problems discussed above. Therefore, it will be necessary to modify the dry powder particles, in order to achieve good dispersion required for accurate dosing. This modification may involve the simple addition of an FCA to the spray dried particles of active material, as discussed above. Alternatively, the FCA may be spray dried together with the active agent.

The co-spray drying of an active agent and another material has been disclosed in the prior art. For example, in WO 96/32149, the co-spray drying of a pharmaceutically active agent and a carrier is proposed. The carrier is said to act as a bulking agent and may be, for example, a carbohydrate or an amino acid. There is little discussion of the spray drying technique, aside from that it involves the spray drying of an aqueous solution and uses conventional spray drying apparatus. The carrier is included in varying amounts and it would appear that this material is evenly distributed throughout the resultant particles. U.S. Pat. No. 6,372,258 discloses the co-spray drying of an active agent and an additive, which is a “minor component” and which is included for conformational stability during spray drying and for improving dispersibility of the powder. The additive materials include hydrophobic amino acids. However, there is no indication of how much of this “minor component” is to be included and there is no indication of how the additive material has the alleged effects. According to the description of this patent, the active agent and additive material are also usually co-spray dried with a carrier or bulking agent, as disclosed in WO96/32149. Once again, there is little discussion of the spray drying process and it appears to be a conventional process using conventional apparatus.

Given the conventional nature of the spray drying process used in the prior art and the rapid rate with which the droplets are dried as a result, the materials being co-spray dried will be uniformly dispersed throughout the particles. Thus, where a hydrophobic amino acid is included in the liquid to be spray dried as a “minor component” it is likely that very little, if any, of the amino acid will be present on the surfaces of the particles.

The inventors have now discovered that spray drying under specific conditions can result in particles with excellent properties which perform extremely well when administered by a DPI for inhalation into the lung. The specific spray drying conditions differ from the conventional spray drying conditions which appear to be used in the abovementioned prior art and have dramatic effects on the particles produced.

In particular, it has been found that manipulating or adjusting the spray drying process can result in a co-spray dried FCA being concentrated on the surfaces of the particles which are produced. This clearly means that the FCA will be better able to reduce the tendency of the particles to agglomerate. When an FCA is not on the surface of a particle but is positioned inside the particle, it will have no beneficial effect on the tendency of the particle to agglomerate.

More specifically, it has now been discovered that the means used to create the droplets which are spray dried is highly significant and will greatly influence the properties of the resultant powder compositions, such as their FPF and FPD. Different means of forming droplets can affect the size and size distribution of the droplets, as well as the velocity at which the droplets travel when formed and the gas flow around the droplets. In this regard, the velocity at which the droplets travel when formed and the gas (which is usually air) flow around the droplets can significantly affect size, size distribution and shape of the resulting dried particles, as well as the distribution of the co-spray dried materials within the particles.

It has been found that it is desirable to control the size and the size distribution of the droplets being formed, as well as the air flow around the droplets. These aspects may be controlled by using alternatives to the conventional nozzles used in spray drying apparatus. In particular, the use of alternative droplet forming means will avoid the use of high velocity air flows.

Thus, according to a first aspect of the present invention, a method of preparing a dry powder composition for inhalation is provided, wherein the active agent is spray dried using a spray drier comprising a means for producing droplets moving at a controlled velocity and of a predetermined droplet size. The velocity of the droplets is preferably controlled relative to the body of gas into which they are sprayed. This can be achieved by controlling the droplets' initial velocity and/or the velocity of the body of gas into which they are sprayed.

It is clearly desirable to be able to control the size of the droplet formed during the spray drying process and the droplet size will affect the size of the dried particle preferably, the droplet forming means also produces a relatively narrow droplet, and therefore particle, size distribution. This will lead to a dry powder formulation with a more uniform particle size and thus a more predictable and consistent FPF and FPD, by reducing the mass of particles with a size above a defined limit.

The ability to control the velocity of the droplet also allows further control over the properties of the resulting particles. In particular, the gas speed around the droplet will affect the speed with which the droplet dries. In the case of droplets which are moving quickly, such as those formed using a two-fluid nozzle arrangement (spraying into air), the air around the droplet is constantly being replaced. As the solvent evaporates from the droplet, the moisture enters the air around the droplet. If this moist air is constantly replaced by fresh, dry air, the rate of evaporation will be increased. In contrast, if the droplet is moving through the air slowly, the air around the droplet will not be replaced and the high humidity around the droplet will slow the rate of drying. As discussed below in greater detail, the rate at which a droplet dries affects various properties of the particles formed, including FPF and FPD.

According to one embodiment of the invention, the method allows dry powder compositions to be prepared which comprise co-spray dried active particles that exhibit a fine particle fraction (<5 μm) of at least 40%. Preferably, the FPF(ED) will be between 60 and 99%, more preferably between 70 and 99%, more preferably between 80 and 99% and even more preferably between 90 and 99%. Furthermore, it is desirable for the FPF(MD) to be at least 40%. Preferably, the FPF(MD) will be between 40 and 99%, more preferably between 50 and 99%, more preferably between 60 and 99%, and more preferably between 70 and 99% and even more preferably between 80 and 99%.

In an important embodiment of the invention, the active agent is co-spray dried with an FCA, the benefits of which are discussed above. Preferred FCAs include all of those mentioned above. Especially useful are those FCAs which have hydrophobic moieties, as these can reduce particle cohesion when they are positioned on the surface of the particles.

Preferably, for delivery to the lower respiratory tract or deep lung, the MMAD of the spray dried active particles is not more than 1.0 μm, and preferably not more than 5 μm, more preferably not more than 3 μm, and may be less than 2 μm, less than 1.5 μm or less than 1 μm. Ideally, at least 90% by weight of the active particles in a dry powder formulation should have a volume diameter (equivalent sphere) of not more than 10 μm, preferably not more than 8 μm more preferably not more than 6 μm, not more than 5 μm not more than 3 μm, not more than 2.5 μm, not more than 2 μm, not more than 1.5 μm, or even not more than 1 μm. Ideally, at least 90% by weight of the active particles in a dry powder formulation should have an aerodynamic diameter of not more than 10 μm, preferably not more than 5 μm, more preferably not more than 3 μm, not more than 2.5 μm, not more than 21 μm, not more than 1.5 μm, or even not more than 1 μm.

In another embodiment, the velocity of droplets at 50 mm from their point of generation is less than 100 m/s, more preferably less than 50 m/s, still more preferably less than 20 m/s and most preferably less than 10 m/s. Preferably, the velocity of the gas used in the generation of the droplets, at 50 mm from the point at which the droplets are generated, is less than 100 m/s, more preferably less than 50 m/s, still more preferably less than 20 m/s and most preferably less than 10 m/s. In an embodiment, the velocity of the droplets relative to the body of gas into which they are sprayed, at 50 mm from their point of generation, is less than 100 m/s, more preferably less than 50 m/s, still more preferably less than 20 m/s and most preferably less than 10 m/s.

Preferably, the velocity of droplets at 10 mm from their point of generation is less than 100 m/s, more preferably less than 50 m/s, still more preferably less than 20 m/s and most preferably less than 10 m/s. Preferably, the velocity of the gas, used in the generation of the droplets, at 10 mm from the point at which the droplets are generated, is less than 100 m/s, more preferably less than 50 m/s, still more preferably less than 20 m/s and most preferably less than 10 m/s. In an embodiment, the velocity of the droplets relative to the body of gas into which they are sprayed, at 10 mm from their point of generation, is less than 100 m/s, more preferably less than 50 m/s, still more preferably less than 20 m/s and most preferably less than 10 m/s.

Preferably, the velocity of droplets at 5 mm from their point of generation is less than 100 m/s, more preferably less than 50 m/s, still more preferably less than 20 m/s and most preferably less than 10 m/s. Preferably, the velocity of the gas, used in the generation of the droplets, at 10 mm from the point at which the droplets are generated is less than 100 m/s, more preferably less than 50 m/s, still more preferably less than 20 m/s and most preferably less than 10 m/s. In an embodiment, the velocity of the droplets relative to the body of gas into which they are sprayed, at 10 mm from their point of generation, is less than 100 m/s, more preferably less than 50 m/s, still more preferably less than 20 m/s and most preferably less than 10 m/s.

In a particularly preferred embodiment of the invention, the means for producing droplets moving at a controlled velocity and of a predetermined size is an alternative to the commonly used nozzles, such as the two-fluid nozzle. In one embodiment, an ultrasonic nebuliser (USN) is used to form the droplets in the spray drying process.

Whilst ultrasonic nebulisers (USNs) are known, these are conventionally used in inhaler devices, for the direct inhalation of solutions containing drug, and they have not previously been widely used in a pharmaceutical spray drying apparatus. It has been discovered that the use of such a nebuliser in a process for spray drying particles for inhalation has a number of important advantages and these have not previously been recognised. The preferred USNs control the velocity of the droplets and therefore the rate at which the particles are dried, which in turn affects the shape and density of the resultant particles. The use of USNs also provides an opportunity to perform spray drying on a larger scale than is possible using conventional spray drying apparatus with conventional types of nozzles used to create the droplets, such as two-fluid nozzles.

As USNs do not require a high gas velocity to generate the droplets, the dryer may provide greater control of the shape, velocity and direction of the plume than is possible with conventional two-fluid, pressure or rotary atomisers. Advantages therefore include reduced drier wall deposition, better controlled and more consistent drying rate. Reduced plume velocity means that smaller drying units are possible.

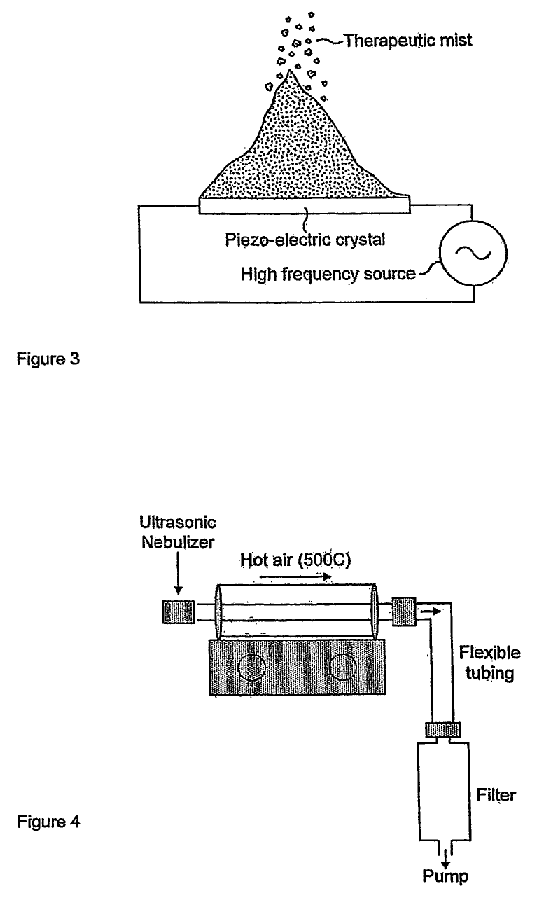

The preferable USNs use an ultrasonic transducer which is submerged in a liquid. The ultrasonic transducer (a piezoelectric crystal) vibrates at ultrasonic frequencies to produce the short wavelengths required for liquid atomisation. In one common form of USN, the base of the crystal is held such that the vibrations are transmitted from its surface to the nebuliser liquid, either directly or via a coupling liquid, which is usually water. When the ultrasonic vibrations are sufficiently intense, a fountain of liquid is formed at the surface of the liquid in the nebuliser chamber. Large droplets are emitted from the apex and a “fog” of small droplets is emitted. A schematic diagram showing how a standard USN works is shown in FIG. 3.

Preferably, the output per single piezo unit (for such a unit oscillating at >1.5 MegaHz) is greater than 1.0 cc/min, greater than 3.0 cc/min, greater than 5.0 cc/min, greater than 8.0 cc/min, greater than 10.0 cc/min, greater than 15.0 cc/min or greater than 20.0 cc/min. Such units should then produce dry particles with at least 90% by weight of the particles having a size of less than 3 μm, less than 2.5 μm or less than 2 μm, as measured by Malvern Mastersizer from a dry powder dispersion unit.

Preferably, the output per single piezo unit (for such a unit oscillating at >2.2 MegaHz) is greater than 0.5 cc/min, greater than 1.0 cc/min, greater than 3.0 cc/min, greater than 5.0 cc/min, greater than 8.0 cc/min, greater than 10.0 cc/min, greater than 15.0 cc/min or greater than 20.0 cc/min. Such units should then produce dry particles with D(90) of less than 3 μm, less than 2.5 μm, or less than 2 μm, as measured by Malvern Mastersizer from a dry powder dispersion unit.

The attractive characteristics of USNs for producing fine particle dry powders for inhalation include: low spray velocity; the small amount of carrier gas required to operate the nebulisers; the comparatively small droplet size and narrow droplet size distribution produced; the simple nature of the USNs (the absence of moving parts which can wear, contamination, etc.); the ability to accurately control the gas flow around the droplets, thereby controlling the rate of drying; and the high output rate which makes the production of dry powders using USNs commercially viable in a way that is difficult and expensive when using a conventional two-fluid nozzle arrangement. This is because scaling-up of conventional spray drying apparatus is difficult and the use of space is inefficient in conventional spray drying apparatus which means that large scale spray drying requires many apparatus and much floor space.

USNs do not separate the liquid into droplets by increasing the velocity of the liquid. Rather, the necessary energy is provided by the vibration caused by the ultrasonic nebuliser.

Furthermore, the USNs may be used to adjust the drying of the droplets and to control the expression of the force control agent on the surface of the resultant particles. Where the active agent itself can act as a force control agent, spray drying with a USN can further help to control the positioning of the hydrophobic moieties so that the effect of including a force control agent can be achieved even without including one.

Thus, as an alternative to the conventional Büchi two-fluid nozzle, an ultrasonic nebuliser may be used to generate droplets, which are then dried within the Büchi drying chamber. In one arrangement, the USN is placed in the feed solution comprising an active agent in a specially designed glass chamber which allows introduction of the cloud of droplets generated by the USN directly into the heated drying chamber of the spray dryer.

The two-fluid nozzle is left in place to seal the hole in which it normally sits, but the compressed air is not turned on. The drying chamber is then heated up to 150° C. inlet temperature, with 100% aspirator setting. Due to the negative pressure of the Büchi system, the nebulised cloud of droplets is easily drawn into the drying chamber, where the droplets are dried to form particles, which are subsequently classified by the cyclone, and collected in the collection jar. It is important that the level of feed solution in the chamber is regularly topped up to avoid over concentration of the feed solution as a result of continuous nebulisation.

Two theories have been developed which describe the mechanism of liquid disintegration and aerosol production in ultrasonic devices (Mercer 1981, 1968 and Sollner 1936). Lang (1962) observed that the mean droplet size generated from thin liquid layers was proportional to the capillary wavelength on the liquid surface. Using the experimentally determined factor of 0.34, the droplet diameter D is given by:
dₚ₌0.34 (8πγ/pf²)^{1/3}
p=solution density g cm³ (water=1)
γ=surface tension dyn cm³ (water=70)
f=frequency (MHz)

This means that for a frequency of 1.7 MHz the calculated droplet size is 2.9 μm and for 2.4 MHz the calculated droplet size is 2.31 μm. Atomisers are also available with frequencies up to 4 MHz with a calculated droplet size of 1.6 μm.

Clearly, this allows the size of the droplets to be accurately and easily controlled, which in turn means that the active particle size can also be controlled (as the dried particle size will depend, to a great extent, on the size of the droplet). Further, the USN provides droplets which are smaller than can be practically produced at a comparative output by a conventional two-fluid nozzle.

Thus, in an embodiment of the present invention, the method of preparing the active particles involves the use of an ultrasonic nebuliser. Preferably, the ultrasonic nebuliser is incorporated in a spray drier.

One type of ultrasonic nebuliser which may be used in the present invention is described in the European Patent Publication No. 0931595 A1. This patent application describes ultrasonic nebulisers which are extremely well suited to putting the present invention into practice.

Despite the fact that the ultrasonic nebulisers disclosed in the patent application are not envisaged as being part of a spray drying apparatus, the nebulisers may be simply and easily incorporated into a spray drier to produce excellent spray dried particles for use in inhalers as indicated above.

The nebulisers disclosed in EP 0931595 A1 are used as air humidifiers. However, the droplets produced are of an ideal size range with a small size distribution for use in a spray drying process. What is more, the nebulisers have a very high output rate of several litres of feed liquid per hour and up to of the order of 60 litres pet hour in some of the devices produced and sold the companies Areco and Sonear. This is very high compared to the two-fluid nozzles used in conventional spray drying apparatus and it allows the spray drying process to be carried out on a commercially viable scale. Other suitable ultrasonic nebulisers are disclosed in U.S. Pat. No. 6,051,257 and in WO 01/49263.

A further advantage of the use of USNs to produce droplets in the spray drying process is that the particles which are produced are small, spherical in shape and are dense. These properties may provide improved dosing. Furthermore, it is thought that the size and shape of the particles produced reduces the drug's device retention to very low levels.

In addition, the USNs can produce comparatively very small droplets relative to other known atomiser types and this, in turn, leads to the production of very small particles. The mean size of the particles produced by USNs tends to be within the range of 0.05 to 5 μm, 0.05 to 3 μm, or even 0.05 to 1 μl. This compares very favourably with the particle sizes which tend to be obtained using conventional spray drying techniques and apparatus, or obtained by milling. Both of these latter methods produce particles with a minimum size of around 1 μm. These advantages associated with the use of USNs are discussed in greater detail below.

The effects of co-spray drying an active agent and an FCA according to the present invention are illustrated in the following discussion of various experiments and the results obtained. The experiments look at various variable factors in the spray drying process and investigate their effects on the nature and performance of the resultant particles.

In the following discussion, reference is made to the following drawings:

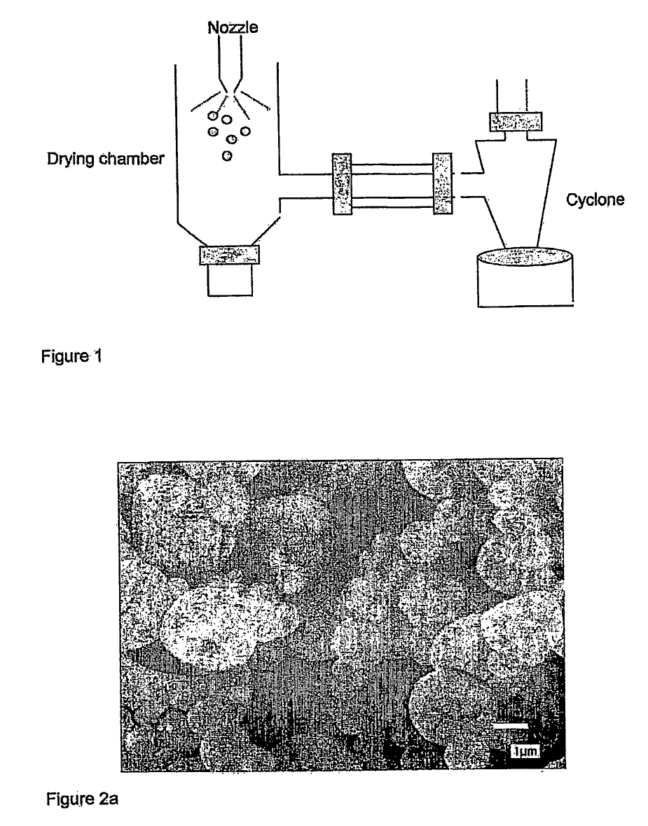

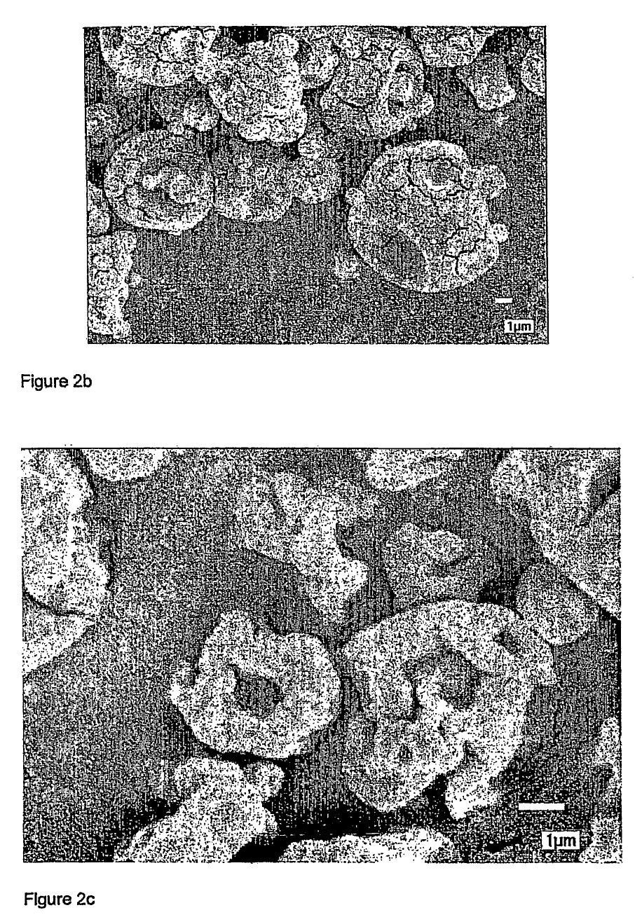

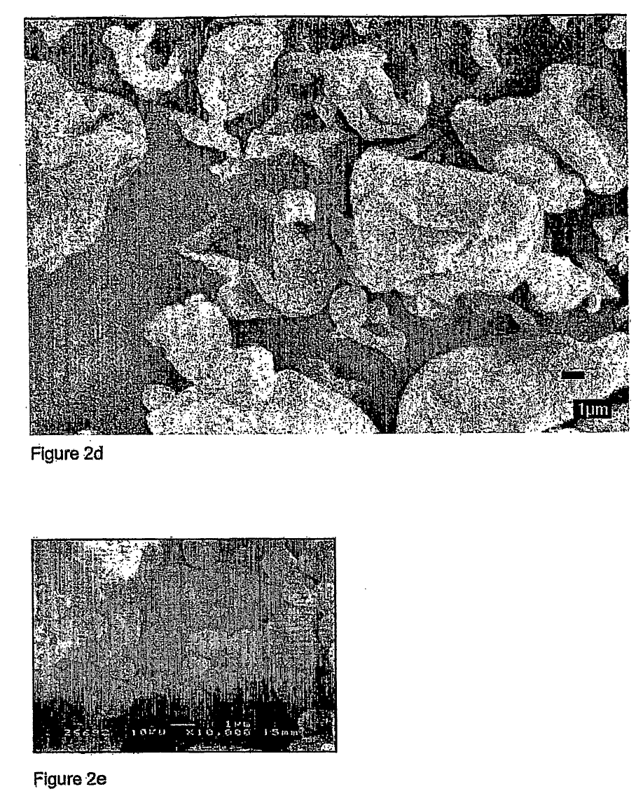

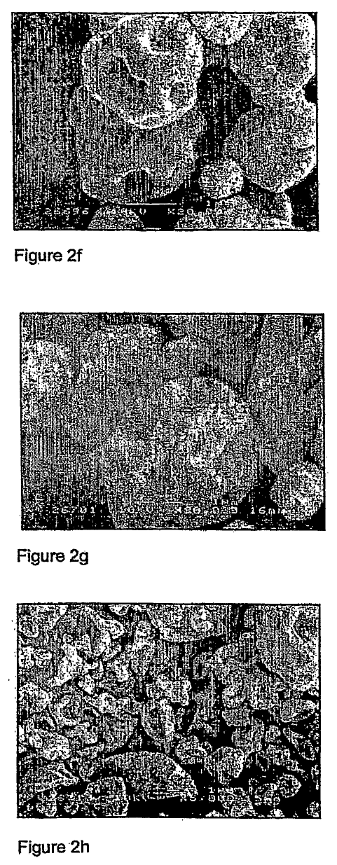

FIG. 1 shows a schematic set-up of a conventional type spray drying apparatus with a two-fluid nozzle; FIGS. 2A-2D are SEM micrographs of two-fluid nozzle spray dried powders which were co-spray dried with increasing amounts of l-leucine (0%, 5%, 25% and 50% w/w), without secondary drying;

FIGS. 2E-2H are SEM micrographs of two-fluid nozzle spray dried powders which were co-spray dried with increasing amounts of l-leucine (2%, 5%, 10% and 50% w/w), after secondary drying;

FIG. 3 shows a schematic diagram of an ultrasonic nebuliser producing fine droplets;

FIG. 4 shows a schematic set-up of a spray drier incorporating an ultrasonic nebuliser;

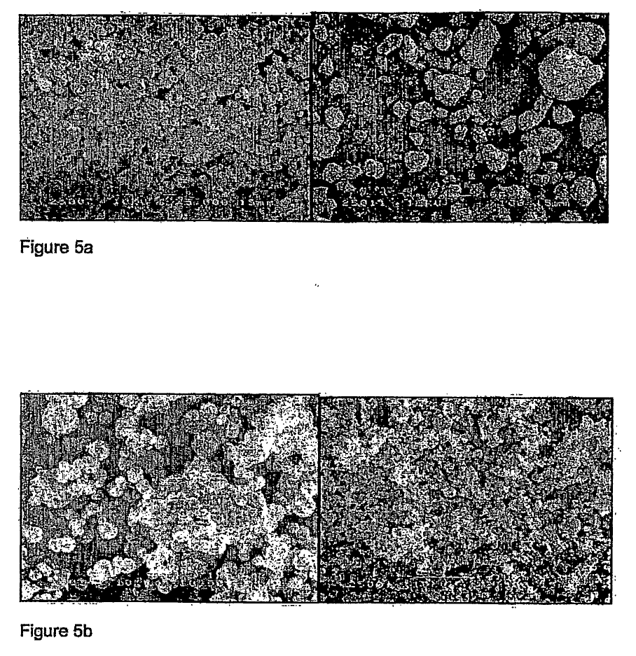

FIGS. 5A and 5B show SEM micrographs of spray dried nebulised heparin alone and with 10% w/w leucine, without secondary drying;

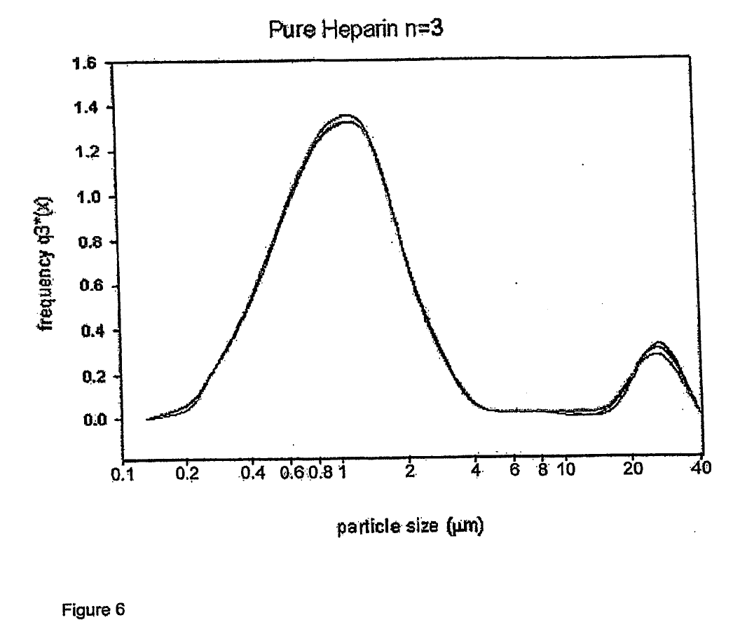

FIG. 6 shows a typical size distribution curve of three repeated tests of spray dried nebulised heparin (with no FCA);

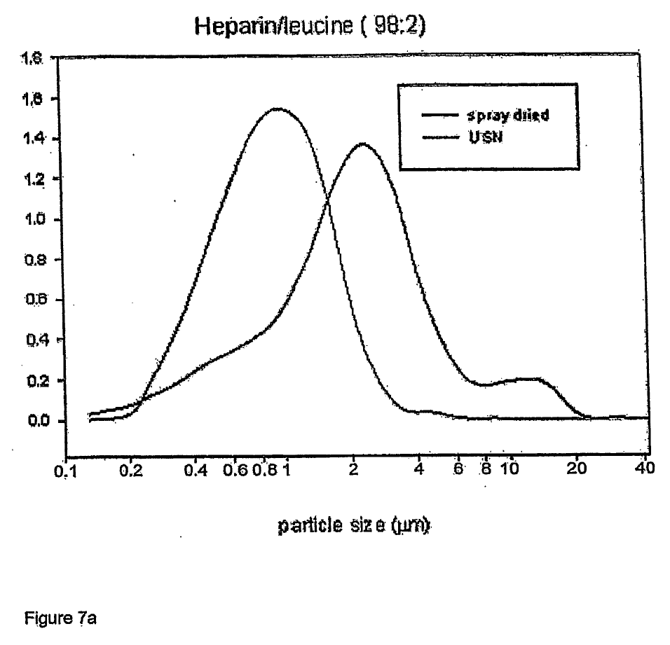

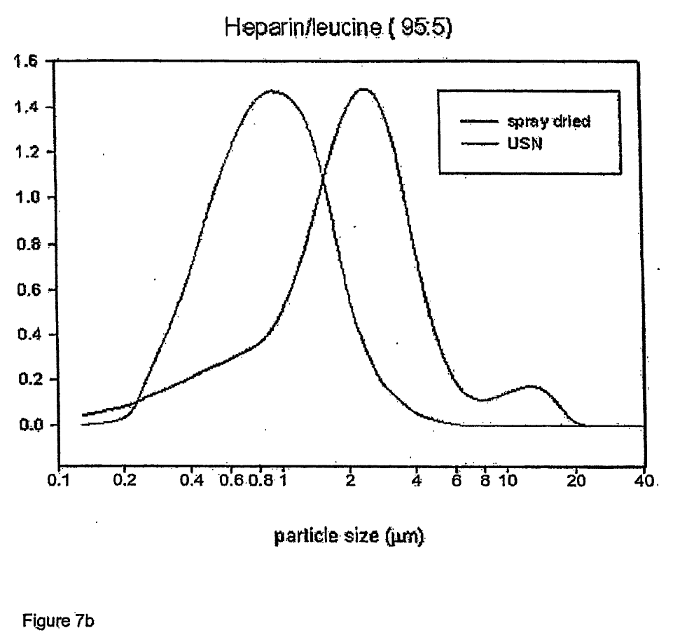

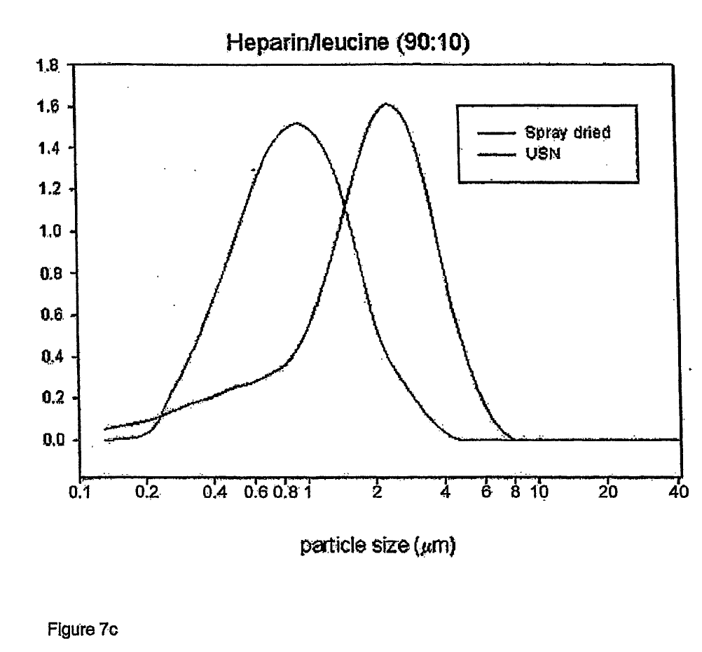

FIGS. 7A-7C show a comparison between particle size distribution curves of two-fluid nozzle spray dried powders and ultrasonic nebulised powders comprising a blend of heparin and leucine (2% w/w, 5% w/w and 10% w/w); and

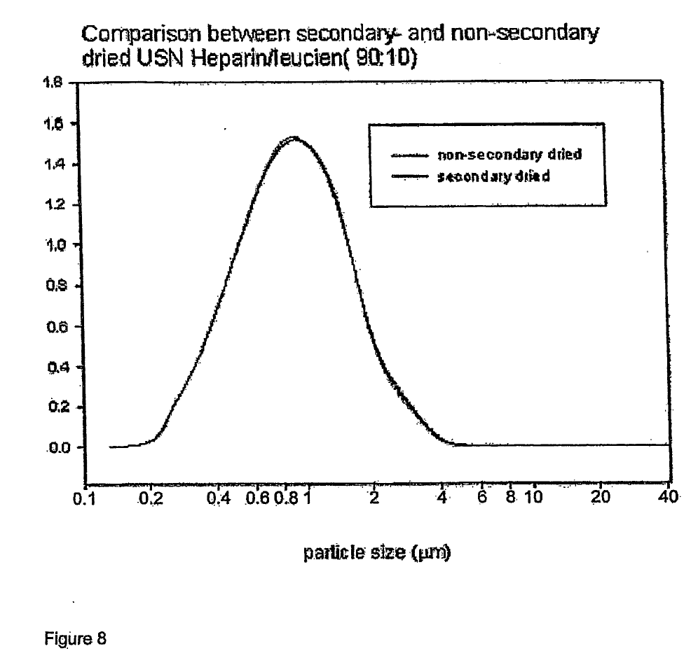

FIG. 8 shows a comparison between particle size distribution curves of secondary dried and not secondary dried powders. The powder used was heparin with leucine (10% w/w).

In the experiments, the active agent used is heparin. The reason for selecting this active agent to illustrate and test the present invention is that heparin is a “sticky” compound and this property tends to have a detrimental effect on the FPF and FPD of the dry powder. Therefore, obtaining good values of FPF and FPD using heparin is an indication that the compositions really do exhibit excellent, improved properties, regardless of the “difficult” nature of the active agent included.

Unless otherwise indicated, the FPF(ED) and FPF(MD) figures given in the following sections of this specification were obtained by firing capsules, filled with approximately 20 mg of material, from a Monohaler into an MSLI, at a flow rate of approximately 90 lpm, or a TSI or rapid TSI at approximately 60 lpm. The fine particle fraction determination from a Miat Monohaler device into an MSLI and a TSI, using the method defined in the European Pharmacopoeia 4th edition 2002, or the ‘Rapid Twin Stage Impinger’ method outlined previously (M. Tservistas et al., A Novel TSI Method for Rapid Assessment of Inhaleable Dry Powder Formulations, Proc. Aerosol Society Conference, Bath, 2001).

The “delivered dose” or “DD”, is the same as the emitted dose or ED (as defined above).

In order to illustrate how the various variable factors of the spray drying process affect the properties of the resultant spray dried particles, firstly the effect of adjusting the solid concentration of active agent was investigated. The active agent was spray dried (without an FCA) using the standard parameters as shown in Table 1, using conventional spray drying apparatus, but the solid concentration of active agent was increased from 1% w/w to 2 and 5% w/w total solids. The effects of these changes on the FPFs were then investigated and the results were as follows.

TABLE 2
FPF (%) less than 5 μm of the emitted dose (ED) for spray dried
heparin using “standard” spray drying parameters

| Description | Test | FPF <5 μm (ED) (%) |
|---|---|---|
| 1% w/w heparin | MSLI | 17.0 |
| 1% w/w heparin | TSI | 20.3 |

The FPF for heparin spray dried alone, that is, without a co-spray dried FCA, using the “standard” spray drying parameters (see Table 1) was 17-20% as shown in Table 2. Testing was done with both an MSLI and a TSI.

TABLE 3
FPF (%) less than 5 μm of ED for heparin spray
dried from increasing solid concentrations

| Description | Test | FPF <5 μm (ED) (%) |
|---|---|---|
| 2% w/w heparin | rTSI | 21.3 |
| 5% w/w heparin | rTSI | 8.3 |

Increasing the solid concentration of heparin from 1% w/w (Table 2) to 5% w/w (Table 3) caused a large reduction in FPF of heparin from approximately 20% FPF to 8.3%, when tested using a rapid TSI. 2% w/w solid content did not seem to have an effect on FPF.

Thus, increasing the solid content of the feed solution did not improve the FPF of the active particles. Increasing the solid content as high as 5% w/w reduced the FPF by more than 10%. Increasing the solid content of a feedstock without changing any of the other parameters generally causes an increase in particle size, as each droplet will have a greater mass of solid which results in larger particles upon drying.

Accordingly, although a solid content of up to 10% w/w active agent, and in some cases as much as 25% w/w active agent, can be used in the present invention, it is preferred for up to 5% w/w, and more preferably up to 2% w/w active agent to be used in the spray drying process of the present invention. It is also preferred for at least 0.05% w/w, and more preferably for at least 0.5% w/w to be employed for practical purposes of production rate.

A further variable factor in the spray drying process is the nature of the feedstock, which may be a solution or a suspension and which can comprise a variety of different solvents or combinations thereof.

In some embodiments, all or at least a proportion of the active agent and/or FCA is or are in solution in the host liquid before being subjected to spray drying. Substantially all of the active agent and FCA can be in solution in the host liquid before being subjected to spray drying.

The active agent is preferably at least 1.5, 2, 4 and, more preferably, at least 10 times more soluble than the FCA in the host liquid at the spraying temperature and pressure. In preferred embodiments, this relationship exists at a temperature between 30 and 60° C. and atmospheric pressure. In other embodiments, this relationship exists at a temperature between 20 to 30° C. and atmospheric pressure, or, preferably, at 20° C. and atmospheric pressure.

The FCA may include one or more water soluble substances. This helps absorption of the substance by the body if the FCA reaches the lower lung. The FCA may include dipolar ions, which may be zwitterions.

Alternatively, the FCA may comprise a substance which is not soluble in water or which is only poorly soluble in water. Where such an FCA is used, it may be advantageous to include further agents to the mixture to be spray dried which will assist solubilising the FCA. For example, the FCA used could be magnesium stearate, which is only slightly soluble in water. However, the addition of an acid will help to solubilise the magnesium stearate and, as the acid will evaporate during the spray drying process, the resultant particles will not suffer from any “contamination” from the acid. Nevertheless, the use of a water soluble FCA is preferred, as the spray drying system is simpler and probably more predictable.

In the present invention, the host liquid preferably includes water. The liquid can employ water alone as a solvent or it may also include an organic co-solvent, or a plurality of organic co-solvents. A combination of water and one or more organic co-solvents is especially useful with active agents and FCAs that are insoluble or substantially insoluble in water alone. Preferred organic co-solvents include methanol, ethanol, propan-1-ol, propanl-2-ol and acetone, with ethanol being the most preferred.

In one embodiment of the present invention, the host liquid consists substantially of water. The use of this host liquid reduces any environmental cost or toxicological complications, or explosive risk. Hence, a host liquid consisting essentially of water provides a significant practical advantage and reduces the process costs.

If an organic solvent is present in the host liquid, it should be selected so that it produces a vapour which is significantly below any explosive or combustion limit. Also, preferably, the spraying composition does not include any blowing agent, such as ammonium carbonate or a halogenated liquid. It may be advantageous to use a non-combustible organic solvent, such as a halogenated solvent. Such aspects are well known to people skilled in the art.

The effect of spray drying an active agent with various organic solvents was evaluated. The “standard” parameters as outlined in Table 1 were used to spray dry heparin, with the only difference being that the heparin was spray dried from 10% w/w organic solvent (propan-1-ol, methanol or ethanol) in water. The results are set out in Table 4.

TABLE 4
FPF (%) less than 5 μm of DD for heparin
spray dried from an organic solvent

| Spray drying | | | |
|---|---|---|---|
| feedstock % w/w | | | FPF <5 μm |
| heparin | Solvent % w/w | Test | (DD) (%) |
| 1 | 10% methanol | MSLI | 2.3 |
| 1 | 10% ethanol | MSLI | 6.2 |
| 1 | 10% propan-1-ol | MSLI | 2.0 |

Spray drying 1% w/w heparin from 10% methanol, ethanol and propan-1-ol resulted in a lowering of FPF (Table 4) from approximately 20% when spray dried from aqueous solvent using identical parameters (shown in Table 2) to 2-6% FPF.

One might expect that adding an organic solvent to the feedstock would cause an increase of the FPF, as a result of a reduction in the viscosity of the feedstock, and a lower energy input being required to generate smaller particles. However, the results obtained from two-fluid nozzle spray drying of heparin from feedstocks containing 10% organic solvent (Table 4) show a reduction in FPF.

Variations in the FPFs are thought to be caused by the effect that the solvent has on the positioning of any hydrophobic moieties of the drug or FCA whilst in the spray drying solution or suspension. The hydrophobic moieties are thought to have the significant force controlling effect. The exposure of a hydrophobic moiety on the surface of a particle is believed to minimise any potential polar forces increasing surface adhesion, such as hydrogen bonds or permanent dipole effects, leaving only the ubiquitous weak London forces. The presence of these hydrophobic moieties on the surface of the particles is therefore important if the cohesion of the powder particles is to be limited, to provide better FPF performance.

When the FCA is in an aqueous solvent, the hydrophobic moieties could be repelled from the interior of the droplet, as the thermodynamics of the system would tend to drive a minimum interaction of these groups with the polar aqueous phase. The positioning of these moieties may therefore be dictated by the nature of the solvent and this, in turn, could affect the positioning of these groups in the eventual spray dried particles. When the aqueous solution of active agent and FCA is spray dried, it may be that the hydrophobic moieties are more likely to be positioned on the surfaces of the particles than if the active agent and FCA are dissolved in an organic solvent, such as ethanol or methanol.

Thus, in one embodiment of the invention, the liquid to be spray dried includes a polar solvent, to encourage the hydrophobic moieties of the material(s) being spray dried to become positioned on the surface of the droplets and then of the spray dried particles.

As a further test of the parameters which might affect the nature of the spray dried particles, an active agent was spray dried using the standard parameters used above (Table 1), but the effect of temperature on the particles produced was investigated by spray drying with inlet temperatures of 75° C. to 220° C. The results are set out in Table 5.

TABLE 5
FPF (%) less than 5 μm of ED for heparin spray
dried using different inlet temperatures

| Inlet | Approx. | | FPF <5 μm |
|---|---|---|---|
| temperature | outlet temperature | Test | (ED) (%) |
| 220° C. | 135° C. | MSLI | 17.5 |
| 75° C. | 35° C. | rTSI | 22.5 |

Thus, it can be seen that spray drying heparin at a higher or lower inlet temperature relative to the “standard” 150° C. normally used did not offer a substantial improvement in FPF.

A preferable range for the inlet temperature is 40° C. to 300° C., preferably 75° C. to 220° C. A preferable range for the outlet temperature is 20° C. to 200° C., preferably 35° C. to 135° C.

The effects of co-spray drying an active agent with varying amounts of the l-leucine, an FCA, from aqueous solution were then studied. Standard Büchi spray drying parameters were used, as shown in Table 1. L-leucine was included in the solution of heparin such that the percentage of l-leucine ranged from 2-50% w/w. The results are set out in Table 6.

1% total solids solutions were sprayed from a two-fluid nozzle into a Büchi spray drier. Blends of heparin and l-leucine were prepared at different weight percentages of l-leucine. Powders of 2%, 5%, 10%, 25% and 50% w/w l-leucine were prepared. The spray drier feed flow rate was 120 ml/hr, the inlet temperature was 150° C., and flush nozzle setting was used. The schematic set-up of the two-fluid nozzle spray drier is shown in FIG. 1.

TABLE 6
FPF (%) less than 5 μm of ED for heparin
co-spray dried with l-leucine

| | Co-spray drying | | |
|---|---|---|---|
| Spray drying | with l-leucine % | | FPF <5 μm |
| feedstock % w/w | w/w | Test | (ED) (%) |
| 1 | 2% | rTSI | 20.0 |
| 1 | 5% | MSLI | 32.8 |
| 1 | 10% | MSLI | 30.8 |
| 1 | 25% | MSLI | 35.4 |
| 1 | 50% | MSLI | 51.7 |

The results show that increasing the percentage of l-leucine included in the feedstock for spray drying resulted in a steady improvement in FPF from approximately 20% FPF with 2% leucine, to 50% FPF with 50% leucine (Table 6).

A further MSLI study was conducted using a powder produced at a feed rate of 300 ml/hr. 20 mg of powder was dispersed in each case. The results set out in Table 7 indicate an improvement of FPF with addition of an FCA, although the FPD does not improve with the addition of more than 10% l-leucine due to the relative reduction of the heparin content.

TABLE 7
MSLI study of co-spray dried heparin
and varying concentrations of leucine

| | | ED | FPF % | FPD |
|---|---|---|---|---|
| Formulation | Test | (mg) | (ED) | (mg) |
| Heparin (0% leucine) | MSLI | 10 | 17 | 1.8 |
| Heparin + leucine (5% w/w) | MSLI | 11 | 33 | 3.6 |
| Heparin + leucine (10% w/w) | MSLI | 13 | 31 | 3.9 |
| Heparin + leucine (25% w/w) | MSLI | 10 | 35 | 3.7 |
| Heparin + leucine (50% w/w) | MSLI | 6 | 52 | 3.0 |

Thus, an improvement in FPF is observed with increasing amounts of FCA. Whilst some improvement is observed at 5% w/w FCA, an FPF of greater than 50% is not achieved until the amount of FCA has been increased to 50% w/w. Smaller amounts of FCA are preferred, in order to reduce the risk of toxicity problems, and may be preferred to reduce the dilution of the active material by FCA to enable dosing to be maximised.

Next, a USN was used to prepare dry powders using a feed solution of an active agent (heparin) alone, and a blend of active agent with 1% to 5% and 10% w/w FCA (l-leucine). The ultrasonic nebuliser output rate was 130 ml/hr. The furnace temperature of the nebulised powders was set at 350° C. FIG. 4 shows a schematic drawing of the ultrasonic set-up.

In order to test the processing of the powders, work was conducted using a Monohaler and a capsule filled with 20 mg powder and fired into a rapid TSI in the manner explained previously. The study used a TSI flow rate of 60 lpm with a cut-off of approximately 5 μm.

Three measurements were made for each blend and the results are summarised below in Table 8, giving the average values of the three sets of results obtained.

TABLE 8
rapid TSI results using the dry powder produced
using a USN with varying amounts of FCA

| | FPF % | FPD |
|---|---|---|
| Formulation | (metered dose) | (mg) |
| Heparin (0% leucine) | 1.1 | 0.22 |
| Heparin + leucine (1% w/w) | 17.4 | 3.5 |
| Heparin + leucine (2% w/w) | 30.2 | 6.0 |
| Heparin + leucine (3% w/w) | 28.6 | 5.7 |
| Heparin + leucine (4% w/w) | 48.4 | 9.7 |
| Heparin + leucine (5% w/w) | 41.5 | 8.3 |
| Heparin + leucine (10% w/w) | 55.8 | 11.8 |

The rapid TSI results using the dry powder produced using the USN indicate a very low aerosolisation efficiency for pure heparin particles, but an improvement appeared in FPF with addition of l-leucine as an FCA.

The poor performance of the pure drug particles compared to those produced using the two-fluid nozzle arrangement (without FCA) is explained by the size of the particles produced by these two different processes. The particles of pure drug generated using the USNs are extremely small (d(50) in the order of 1 μm) compared to those prepared using the two-fluid nozzle arrangement (d(50) in the order of 2.5 μm). Without an FCA, the smaller particles produced using the USN exhibit a worse FPF than the larger particles produced by the two-fluid nozzle, due to the increased surface free energy of the smaller particles.

The effect of including an FCA appears to be magnified when the spray drying apparatus includes a USN. Thus, it will not be necessary to include amounts of up to 50% w/w of FCA in the feed solution, as suggested from the experiment involving a conventional spray drying process, as discussed above. Rather, it has been found that excellent FPF values are achieved when no more than 20% w/w FCA is included. Preferably, no more than 10% w/w, no more than 8% w/w, no more than 5% w/w, no more than 4% w/w, no more than 3% w/w, no more than 2% w/w, no more than 1% w/w, or no more that 0.5% w/w FCA is spray dried using a USN. The amount of FCA included may be as low as 0.1% w/w, especially where the active agent is not able to act as an FCA itself.

Where the spray drying takes place under “standard” parameters and using conventional spray drying apparatus, it has been found that spray drying an active agent with an FCA can lead to non-spherical particle morphology. At low concentrations of FCA, the surfaces of the particles show dimples or depressions. As the amount of co-spray dried FCA is increased, these dimples become more extreme, with the particles eventually having a shrivelled or wrinkled surface, the net effect therefore is the formulation of less dense particles.

The morphology of the particles prepared using the two-fluid nozzles and the USNs was viewed using scanning electron micrographs (SEMs).

SEM micrographs of two-fluid nozzle spray dried powders (FIGS. 2A-D) illustrate a clear relationship between the increasing percentage of l-leucine and an increasingly dimpled or wrinkled surface of the particles. The particles with the highest l-leucine content appear to be extremely wrinkled and, in selected cases, may even burst as an extreme result of “blowing”, a phenomenon whereby the particles form a shell or skin which inflates due to the evaporation of the solvent, creating a raised internal vapour pressure and then may collapse or burst.

Droplets from the two-fluid nozzle are initially dried at a relatively high rate during spray drying. This creates a viscous layer of material around the exterior of the liquid droplet. As the drying continues, the viscous layer is firstly stretched (like a balloon) by the increased vapour pressure inside the viscous layer as the solvent evaporates. The solvent vapour diffuses through the growing viscous layer until it is exhausted and the viscous layer then collapses, resulting in the formation of craters in the surface or wrinkling of the particles.

The net effect of the inflation, stretching of the skin and deflation is the creation of significant numbers of craters and wrinkles or folds on the particle surface, which consequently results in a relatively low density particle which occupies a greater volume than a smooth-surfaced particle.

This change in the surface morphology of these co-spray dried particles may contribute to reduced cohesion between the particles. Particles of pure active material are generally spherical in shape, as seen in FIG. 2A. It has been argued that increased particle surface roughness or rugosity, such as is caused by surface wrinkles or craters, results in reduced particle cohesion and adhesion by minimising the surface contact area between particles.

FIG. 5A shows SEM micrographs of USN spray dried heparin alone, whilst FIG. 5B shows SEM micrographs of USN spray dried heparin with 10% leucine.

As can be clearly seem from the SEMs, the shape of particles formed by co-spray drying an active agent and leucine using a USN differs to that of particles formed by co-spray drying heparin and leucine using a conventional two-fluid nozzle spray drying technique.

The SEMs of pure heparin generated using a USN show that the particles have a size of generally less than 2 μm. The SEMs also show that these particles tend to form “hard” agglomerates of up to 200 μm.

The SEMs of heparin and leucine generated using a USN show that the primary particles produced are of the same size as the pure heparin particles. However, these particles are discrete and agglomerates are less evident and less compacted in nature.

What is more, the distinctive dimples or wrinkles observed on the surface of the particles prepared by co-spray drying heparin and leucine using a two-fluid nozzle spray drier (FIGS. 2A-2D) are less evident when the particles are spray dried using a USN. Despite this, the co-spray dried particles formed using a USN still have an improved FPF and FPD over particles formed in the same way but without the FCA. In this case, this improvement cannot be attributed to the shape of the particles, nor is it due to any change in density or rugosity. It may be attributed to the leucine concentration at the particle surfaces.

It is believed that the FCA concentration at the surface of a solid particle from spray drying is governed by several factors. These include the concentration of FCA in the solution which forms the droplets, the relative solubility of the FCA compared to the active agent, the surface activity of the FCA, the mass transport rate within the drying droplet and the speed at which the droplets dry. If drying is very rapid it is thought that the FCA content at the particle's surface will be lower than that for a slower drying rate. The FCA surface concentration may be determined by the rate of FCA transport to the surface, and its precipitation rate, during the drying process.

As mentioned above, high gas flow speed rates around the droplets can accelerate drying and it is thought that, because the gas speed around droplets formed using a USN is low in comparison to that around droplets formed using conventional two-fluid nozzles, droplets formed using a USN dry more slowly than those produced by using conventional two-fluid nozzles. The FCA concentration on the surface of droplets and dried particles produced using a USN can be higher as a result. It is considered that these effects reduce the rate of solvent evaporation from the droplets and reduce “blowing” and, therefore, are responsible for the physically smaller and smoother primary particles that are observed (Kodas, T. T. and Hampden Smith, M., 1999, Aerosol Processing of Materials, 440). In this last regard, and as previously noted, droplets formed by the two-fluid nozzle system have rapid air flow around them and they, therefore, dry very rapidly and markedly exhibit the effects of blowing.

It is also speculated that the slower drying rate which is expected when the droplets are formed using USNs allows the FCA to migrate to the surface of the droplet during the drying process. This migration may be further assisted by the presence of a solvent which encourages the hydrophobic moieties of the FCA to become positioned on the surface of the droplet. An aqueous solvent is thought to be of assistance in this regard.

With the FCA being able migrate to the surface of the droplet so that it is present on the surface of the resultant particle, it is clear that a greater proportion of the FCA which is included in the droplet will actually have the force controlling effect (as the FCA must be present on the surface in order for it to have this effect). Therefore, it also follows that the use of USNs has the further advantage that it requires the addition of less FCA to produce the same force controlling effect in the resultant particles, compared to particles produced using conventional spray drying methods.

Naturally, where the active agent itself has hydrophobic moieties which can be presented as a dominant composition on the particle surface, excellent FPF and FPD values may be achieved with little or no separate FCA. Indeed, in such circumstances, the active agent itself acts as an FCA, because of the arrangement of its hydrophobic moieties on the surfaces of the particles.

The movement of the FCA during the drying step of the spray drying process will also be affected by the nature of the solvent used in the host liquid. As discussed above, an aqueous solvent is thought to assist the migration of the hydrophobic moieties to the surface of the droplet and therefore the surface of the resultant particle, so that the force controlling properties of these moieties is maximised.

Finally, it should also be noted that the particles produced using the USNs appear to have a higher density than the wrinkled particles produced using the two-fluid nozzles. It can is actually be advantageous not to produce severely dimpled or wrinkled particles, as these can yield low density powders, with very high voidage between particles. Such powders occupy a large volume relative to their mass as a consequence of this form, and can result in packaging problems, i.e., much larger blisters or capsules are required for a given mass of powder. High density powders may, therefore, be of benefit, for example, where the dose of active agent to be administered is high.

Advantageously, powders according to the present invention have a tapped density of at least 0.1 g/cc, at least 0.2 g/cc, at least 0.3 g/cc, at least 0.4 g/cc or at least 0.5 g/cc.

It has previously been speculated that this particle morphology may even help the particles to fly when they are expelled for the inhaler device. However, despite this speculation relating to the benefits of the irregular shapes of the particles to be inhaled, the inventors actually feel that the chemical nature of the particle surfaces may be even more influential on the performance of the particles in terms of FPF, ED, etc. In particular, it is thought that the presence of hydrophobic moieties on the surface of particles is more significant in reducing cohesion than the presence of craters or dimples. Therefore, contrary to the suggestion in the prior art, it is not necessary to seek to produce extremely dimpled or wrinkled particles in order to provide good FPF values.

Next, the effect of spray drying an active agent with various excipients was investigated. Standard spray drying parameters as shown in Table 1 were used and the various excipients tested were lactose, dextrose, mannitol and human serum albumin (HSA). The excipients were co-spray dried with heparin from aqueous solution. Between 5-50% w/w of the excipients were included, with total solid content not exceeding 1% w/w of the solution.

TABLE 9
FPF (%) less than 5 μm of ED for heparin
co-spray dried with excipients

| Spray drying | Co-spray drying | | FPF <5 μm |
|---|---|---|---|
| feedstock % w/w | excipient % w/w | Test | (ED) (%) |
| 1 | 5% lactose | rTSI | 7.0 |
| 1 | 20% lactose | rTSI | 5.3 |
| 1 | 50% lactose | rTSI | 10.3 |
| 1 | 5% dextrose | rTSI | 11.0 |
| 1 | 50% dextrose | rTSI | 1.7 |
| 1 | 5% mannitol | rTSI | 14.0 |
| 1 | 20% mannitol | rTSI | 11.3 |
| 1 | 5% HSA | rTSI | 34.0 |
| 1 | 50% HSA | rTSI | 28.0 |

Inclusion of lactose (5-50%), dextrose (5-50%) and mannitol (5-20%) did not improve the FPF (Table 9). In fact, for all of these excipients, FPFs fell to below the “standard” 20% for spray dried heparin. However, inclusion of 5% HSA gave an improvement.

As the presence of the HSA in the active particle clearly reduces the particle cohesion, thereby increasing the FPF, HSA may be considered, for the purpose of the present invention, to be an FCA. However, in some embodiments of the invention, the FCA used is preferably not HSA.

It is believed that the ability of HSA to act as an FCA when co-spray dried as described above may be due to the arrangement of the hydrophobic moieties of the HSA on the surface of the spray dried particles. As discussed above, the positioning of hydrophobic groups on the surface of the spray dried particles is considered to be very important and can affect the cohesiveness and adhesiveness of the particles in a dry powder formulation. Proteins, such as HSA, tend to have hydrophobic parts of their constituent amino acids which allow them to act as FCAs under the appropriate conditions. Indeed, in one embodiment of the present invention, where the active agent is a protein, under the correct spray drying conditions, the active agent may itself act as an FCA, thereby avoiding the need to spray dry the protein with a separate FCA. The protein would preferably be spray dried in a manner that will allow the hydrophobic moieties to be arranged on the surface of the resultant particles. Therefore, the host solution is preferably an aqueous solution. Additionally, the drying of the particles should occur at a rate which allows the movement of the hydrophobic moieties or retention of the moieties at the surface.

In one embodiment of the present invention, the active agent is not co-spray dried with a carrier or excipient material. In another embodiment, the active agent is not co-spray dried with a carrier or excipient material unless that material has hydrophobic moieties (which allow it to act as a FCA).

Thus, in another embodiment of the present invention, a method is provided for producing spray dried particles comprising a protein as both the active agent and an FCA. The particles exhibit FPF(ED) and FPF(MD) which is better than those exhibited by conventionally spray dried particles of protein, as a result of the hydrophobic moieties arranged on the surface of the spray dried particles according to the present invention.

In a particle size study, the particle size of the spray dried particles formed using the USN was analysed. The dry powders were dispersed at 4 bar in Sympatec particle sizer (Helos dry dispersed). The values of d(10), d(50) and d(90) of the ultrasonic nebulised powders were measured and are indicated in Table 10 (10% by volume of the particles are of a size, measured by Sympatec, that is below the d(10) value, 50% by volume of the particles are of a size, measured by Sympatec, that is below the d(50) value and so on). The values are an average of three measurements.

In addition, the percentage mass of particles with a size of less than 5 μm was obtained from the particle size data and is expressed as FPF.

TABLE 10
Particle size study of spray dried particles
using USN, without secondary drying

| | d(10) | d(50) | d(90) | FPF % |
|---|---|---|---|---|
| Formulation | (μm) | (μm) | (μm) | (<5 μm) |
| Heparin (0% leucine) | 0.43 | 1.07 | 4.08 | 90.52 |
| Heparin + leucine (1% w/w) | 0.41 | 0.90 | 1.79 | 99.97 |
| Heparin + leucine (2% w/w) | 0.41 | 0.89 | 1.75 | 100 |
| Heparin + leucine (3% w/w) | 0.41 | 0.88 | 1.71 | 100 |
| Heparin + leucine (4% w/w) | 0.41 | 0.86 | 1.71 | 100 |
| Heparin + leucine (5% w/w) | 0.41 | 0.90 | 1.84 | 100 |
| Heparin + leucine (10% w/w) | 0.41 | 0.89 | 1.76 | 100 |

FIG. 6 shows a typical size distribution curve of three repeated tests of pure heparin powder generated using an ultrasonic nebuliser. The main peak represents the size of the individual active particles, ranging between 0.2 μm and 4.5 μm in diameter. The second, smaller peak between diameters of 17 to 35 μm represents agglomerates of active particles

Sympatec particle sizing (Helos dry dispersed) results showed that ultrasonic nebulised powders have a narrower size distribution and smaller mean particle size than the two-fluid nozzle spray dried powders.

FIG. 7A shows a comparison between particle size distribution curves of two-fluid nozzle spray dried powders and ultrasonic nebulised powders comprising a blend of heparin with 2% leucine w/w.

FIG. 7B shows a comparison between particle size distribution curves of two-fluid nozzle spray dried powders and ultrasonic nebulised powders comprising a blend of heparin with 5% leucine w/w.

FIG. 7C shows a comparison between particle size distribution curves of two-fluid nozzle spray dried powders and ultrasonic nebulised powders comprising a blend of heparin with 10% leucine w/w.

These figures also show a gradual disappearance of the second peak, indicating that the incidence of agglomerates is reduced as the amount of co-spray dried FCA is increased.

For the USN spray dried material, agglomerate peaks disappear under the same test conditions when >3% leucine is added. For the two-fluid nozzle spray dried material, agglomerate peaks disappear under the same test conditions when >10% leucine is added. This indicates that adding leucine as an FCA reduces the strength of the agglomerates in heparin powder. It further suggests that ultrasonic nebulised materials de-agglomerate more easily with a lower FCA content. This may be related to the surface concentration of the FCA, as mentioned above.

The SEM images of ultrasonic nebulised powders (FIGS. 5A and 5B) also support the finding that addition of leucine facilitates aerosolisation. SEMs of pure heparin showed that although heparin primary particles are generally <2 μm large distinct agglomerates are formed. The SEMs of all of the powders comprising heparin and leucine show that the primary particle size is still <2 μm, but the large hard agglomerates are not evident.

It can be seen that particles formed using a spray drying process involving an ultrasonic nebuliser have been found to have a greater FPF than those produced using a standard spray drying apparatus, for example with a two-fluid nozzle configuration.

What is more, the particles formed using a spray drying process using a USN have been found to have a narrower particle size distribution than those produced using a standard spray drying apparatus, for example with a two-fluid nozzle configuration.

Studies of the particles produced by spray drying using USNs have led to the discovery that the bulk density of ultra-fine drug powders can be beneficially increased whilst also improving aerosolisation characteristics. This finding is contrary to conventional thinking and in marked contrast to the prior art approaches to improving aerosolisation, whereby drug particles and formulations are prepared having reduced density. Whilst low density particles can improve aerosolisation, they place significant limitations on payload mass which can be delivered as a single inhalation. For example, a size 3 capsule (the type of capsule used in Cyclohaler (trade mark), Rotahaler (trade mark) and many other capsule-based DPIs) which conventionally holds 20 mg of formulated powder might only accommodate 5 mg or less of a low density material.

The significance and commercial benefit of high density or densified powder articles is that it provides the potential to deliver increased powder payloads in smaller volumes. For example, a size 3 capsule which conventionally holds a 20 mg payload, may be able to accommodate up to 40 mg of a higher density powder formulation and an Aspirair (trade mark) blister designed to hold a 5 mg payload may be used to hold 15 mg of a higher density powder such as that which may be produced using the present invention. This is particularly important for drugs requiring high dose delivery, including, for example, heparin, where doses in the region of 40-50 mg may be required. It should be possible to incorporate this dose in the form of a high density powder into a blister or capsule which holds just 20-25 mg of a standard density powder.

Using the above described spray drying process using a USN, the final density of particles comprising active agent and FCA (heparin and leucine) has been increased by controlled atomisation and drying. The ability to increase density, as noted above, provides an opportunity to increase drug payloads filled into a unit blister or capsule whilst, in this case, raising FPD from 20% for conventionally spray dried heparin to 70% for heparin and an FCA spray dried according to the present invention.

The key to improved aerosolisation in a denser particle is the presence of an FCA on the surfaces of the particles, without which the benefits of densification cannot be realised. The process by which densification is brought about is also critical in terms of the spatial positioning of the FCA on the drug particle surface. The aim is always to provide the maximum possible surface presence of FCA in the densified drug composite. In the case of the spray drying according to the present invention, conditions are selected to provide FCA surface enrichment of resultant drug particles.

In a further experiment, ultrasonic nebulised formulations comprising clomipramine or heparin with 5% w/w leucine were prepared and were tested in Aspirair (trade mark) and Monohaler (trade mark) devices.

The heparin formulation was produced, using a spray drying system according to the present invention, as described above. This system comprises an ultrasonic nebulisation unit, a gas flow for transporting the droplets nebulised into a heated tube to dry the droplets, and a filtration unit for collecting the dried particles.

An aqueous solution of the heparin was made containing 1% w/w relative to the water. Leucine, an FCA, was added to this in an amount sufficient to make 5% w/w relative to the heparin.

The solution was nebulised with a frequency of 2.4 MHz and guided through the tube furnace with furnace surface temperature heated to approximately 300° C., after which the dried powder was collected. The gas temperature was not measured, but was substantially less than this temperature. Malvern Mastersizer (dry powder) particle size measurement gave a d(50) of 0.8 μm.

The clomipramine hydrochloride formulation was produced from the original powder, using the same spray drying system as noted above for heparin.

An aqueous solution of the clomipramine hydrochloride was made containing 2% w/w relative to the water. Sufficient leucine was added to make 5% w/w relative to the drug. The solution was nebulised with a frequency of 2.4 MHz and guided through the tube furnace with furnace surface temperature heated to approximately 300° C., after which the dried powder was collected. The gas temperature was not measured, but was substantially less than this temperature. Malvern (dry powder) particle size measurement gave a d(50) of 1.1 μm

The Malvern particle size distributions show that both the heparin and the clomipramine hydrochloride have very small particle sizes and size distributions. The d(50) values are 0.8 μm for heparin and 1.1 μm for clomipramine hydrochloride. The modes of the distribution graph are correspondingly 0.75 and 1.15. Further, the spread of the distributions is relatively narrow, with d(90) values of 2.0 μm and 2.5 μm respectively, which indicates that substantially all of the powder by mass is less than 3 μm and, in the case of the heparin, less than 2 μm. Heparin shows a smaller particle size and size distribution than clomipramine hydrochloride, probably due to lower concentration in the original solution.

Approximately 3 mg and 5 mg of the heparin formulation and 2 mg of the clomipramine hydrochloride formulation were then loaded and sealed into foil blisters. These were then fired from an Aspirair device into an NGI with air flow set at 90 l/min. The results for the heparin are based upon a cumulative of 5 fired blisters. Only 1 blister shot was fired for each clomipramine hydrochloride NGI.

Approximately 20 mg of the heparin or the clomiptamine hydrochloride formulations were loaded and sealed into size 3 capsules. The clomipramine hydrochloride capsules were gelatine capsules and the capsules used for the heparin formulation were HPMC capsules (hydroxypropylmethyl cellulose). These capsules were then fired using the Monohaler device into a NGI with an air flow set at 90 l/min. The performance data are summarised as follows, the data being an average of 2 or 3 determinations:

TABLE 11
Powder performance study of drug and 5% leucine
dispensed using Aspirair (trade mark)

| | MD | ED | FPD | FPF % | FPF % | FPF % | FPF % |
|---|---|---|---|---|---|---|---|
| Aspirair | (μm) | (μm) | (μm) | (<5 μm) | (<3 μm) | (<2 μm) | (<1 μm) |
| Heparin | 1969 | 1870 | 1718 | 92 | 83 | 69 | 39 |
| 3 mg | | | | | | | |
| Heparin | 3560 | 3398 | 3032 | 89 | 78 | 60 | 31 |
| 5 mg | | | | | | | |
| Clomipra- | 1739 | 1602 | 1461 | 91 | 81 | 62 | 28 |
| mine 2 mg | | | | | | | |

TABLE 12
Powder performance study of drug and 5% leucine
dispensed using Aspirair (trade mark)

| | | Throat | Blister | Device |
|---|---|---|---|---|
| Aspirair | MMAD | (%) | (%) | (%) |
| Heparin 3 mg | 1.30 | 5 | 2 | 2 |
| Heparin 5 mg | 1.57 | 6 | 2 | 2 |
| Clomipramine 2 mg | 1.56 | 4 | 3 | 5 |

TABLE 13
Powder performance study of drug and 5% leucine
dispensed using Monohaler (trade mark)

| | MD | ED | FPD | FPF % | FPF % | FPF % | FPF % |
|---|---|---|---|---|---|---|---|
| Monohaler | (μm) | (μm) | (μm) | (<5 μm) | (<3 μm) | (<2 μm) | (<1 μm) |
| Heparin 20 mg | 14201 | 12692 | 10597 | 83 | 70 | 54 | 29 |
| Clomipramine 20 mg | 18359 | 16441 | 12685 | 77 | 56 | 37 | 19 |

TABLE 14
Powder performance study of drug and 5% leucine
dispensed using Monohaler (trade mark)

| | | Throat | Blister | Device |
|---|---|---|---|---|
| Monohaler | MMAD | (%) | (%) | (%) |
| Heparin 20 mg | 1.72 | 6 | 5 | 6 |
| Clomipramine 20 mg | 2.38 | 10 | 1 | 9 |

The device retention in the Aspirair device was surprisingly low (between 2-5%) for both drug formulations. This was especially low given the small particle sizes used and the relatively high dose loadings used. For example, the clomipramine hydrochloride exhibited device retention in the Aspirair device of 5% and a small d(50) of 1.1 μm. In comparison, clomipramine hydrochloride co-jet milled with 5% leucine with a d(50) of 0.95 μm gave a device retention of 23% under otherwise similar circumstances. Heparin gave very low device retention in Aspirair with a d(50) of 0.8 μm and there did not appear to be a difference in device retention using the 3 mg or 5 mg filled blisters.

When using the Monohaler device to dispense the formulations, the device retention was higher than observed when the Aspirair device was used. However, device retention of respectively 6% for heparin and 9% for clomipramine hydrochloride still appears to be relative low for a formulation that comprises>90% ultrafine drug.

Throat retention was also very low for both drug formulations. When the formulations were dispensed using the Aspirair device, it was as low as 4%. With the Monohaler device, the results show slightly higher throat retention (between 6-10%).

It has previous been argued that as particle size is reduced, powder surface free energy and hence powder adhesivity and cohesivity increases. This would be expected to result in increased device retention and poor dispersion. Such adhesivity and cohesivity, and hence device retention/poor performance has been shown to be reduced by addition of FCA on the surface of the drug particles (or the drug and excipient particles, as appropriate). In the Aspirair device, it is believed that a certain degree of adhesivity and cohesivity is desirable to prolong lifetime in the vortex, yielding a slower plume, but adhesivity and cohesivity should not be so high as to result in high device retention. Consequently a balance of particle size, adhesivity and cohesivity is believed to be required to achieve an optimum performance in the Aspirair device.

The dispersion results for both powders were also excellent when using the Monohaler device.

It is believed that the results indicate that the ultrasonic nebulising process results in a most effective relative enrichment of FCA concentration at the particle surface. The surface enrichment is dependent upon the rate of the FCA transport to the surface, the size of the particle, and its precipitation rate, during the drying process. This precipitation rate is related to the slow drying of the particles in this process. The resulting effect is that the particle surface is dominated by the hydrophobic aspects of the FCA. This presents a relatively low surface energy of the powder despite its small particle size and high surface area. It therefore appears that the addition of an FCA is having a superior influence to adhesivity and cohesivity and hence the device retention and dispersion.

The inclusion of leucine appears to provide significant improvements to the aerosolisation of heparin and clomipramine hydrochloride, and should make both drugs suitable for use in a high-dose passive or active device.

One would expect to get similar results to those shown above using USNs when using other means which produce low velocity droplets at high output rates. For example, further alternative nozzles may be used, such as electrospray nozzles or vibrating orifice nozzles. These nozzles, like the ultrasonic nozzles, are momentum free, resulting in a spray which can be easily directed by a carrier air stream. However, their output rate is generally lower than that of the USNs described above.

Another attractive type of nozzle for use in a spray drying process is one which utilises electro-hydrodynamic atomisation. A tailor cone is created at a fine needle by applying high voltage at the tip. This shatters the droplets into an acceptable monodispersion. This method does not use a gas flow, except to transport the droplets after drying. An acceptable monodispersion can also be obtained utilising a spinning disc generator. The nozzles such as ultrasonic nozzles, electrospray nozzles or vibrating orifice nozzles can be arranged in a multi nozzle array, in which many single nozzle orifices are arranged in a small area and facilitate a high total throughput of feed solution. The ultrasonic nozzle is an ultrasonic transducer (a piezoelectric crystal). If the ultrasonic transducer is located in an elongate vessel the output may be raised significantly.

When active particles are produced by spray drying, some moisture will remain in the particles. This is especially the case where the active agent is temperature sensitive and does not tolerate high temperatures for the extended period of time which would normally be required to remove further moisture from the particles.

Therefore, in a further embodiment of the present invention, the method of preparing a dry powder composition further comprises a step of adjusting the moisture content of the particles. Adjusting the moisture content of the spray dried particle allows fine-tuning of some of the properties of the particles. The amount of moisture in the particles will affect various particle characteristics, such as density, porosity, flight characteristics, and the like.

In one embodiment, the moisture adjustment or profiling step involves the removal of moisture. Such a secondary drying step preferably involves freeze-drying, wherein the additional moisture is removed by sublimation. An alternative type of drying for this purpose is vacuum drying. Generally, the secondary drying takes place after the active agent has been co-spray dried.

The secondary drying step has two particular advantages. Firstly, it can be selected so as to avoid exposing the pharmaceutically active agent to high temperatures for prolonged periods. Furthermore, removal of the residual moisture by secondary drying can be significantly cheaper than removing all of the moisture from the particle by spray drying. Thus, a combination of spray drying and freeze-drying or vacuum drying is economical and efficient, and is suitable for temperature sensitive pharmaceutically active agents.

In order to establish the effect of secondary drying of the powders, samples of active agent alone and of a combination of active agent (heparin) and an FCA (leucine 10% w/w), were secondary dried at 50° C. under vacuum for 24 hours.

The results set out in Table 15 indicate the secondary drying step further raised the FPF and FPD, when they are compared to the results in Table 10, which relates to equivalent particles which have not undergone secondary drying.

TABLE 15
rapid TSI results using the dry powder produced using a
USN with varying amounts of FCA, after secondary drying

| | FPF % | FPD |
|---|---|---|
| Formulation | (metered dose) | (mg) |
| Heparin (0% leucine) | 4.1 | 0.82 |
| Heparin + leucine (10% w/w) | 70.8 | 14.2 |

In a later stage experiments have been conducted on samples of active agent (heparin) and an FCA (leucine 5% w/w), were secondary dried at 40° C. under vacuum for 24 hours.

Particle size tests were also conducted to show the effect of secondary drying. The particle size of the spray dried particles formed using the USN was analysed. The dry powders were dispersed at 4 bar in a Helos disperser. The powders were secondary dried over 24 hours under vacuum.

The values of FPF <5 μm and d(10), d(50) and d(90) of the ultrasonic nebulised powders were measured and are indicated in Table 16.

TABLE 16
Particle size study of spray dried particles
using USN, after secondary drying

| | | | | FPF % |
|---|---|---|---|---|
| Formulation | d(10) | d(50) | d(90) | (<5 μm) |
| Heparin (0% leucine) | 0.44 | 1.06 | 2.93 | 92.35 |
| Heparin + leucine (10% w/w) | 0.40 | 0.87 | 1.77 | 100 |

Thus, by comparing the results in Table 16 with those of Table 10, one can see that secondary drying particles did not result in any significant change in particle size, both for active agent alone and for a blend of active agent and FCA.

FIG. 6 shows a comparison between particle size distribution curves of secondary dried and not secondary dried powders. The powder used was heparin with 10% leucine w/w. There is no significant difference between the curves, illustrating that secondary drying does not have an effect on particle size.

Then, in order to establish whether the effect of secondary drying varied between particles produced using a USN and a two-fluid nozzle, the particle size study of secondary drying with spray dried particles formed using the USN was repeated but using a two-fluid nozzle spray drier. Once again, the powders were secondary dried over 24 hours under vacuum. Values of FPF <5 μm and d(10), d(50) and d(90) of the spray dried powders are indicated in Table 17 below.

TABLE 17
Particle size study of two-fluid nozzle spray
dried particles after secondary drying

| | | | | FPF % |
|---|---|---|---|---|
| Formulation | d(10) | d(50) | d(90) | (<5 μm) |
| Heparin + leucine (2% w/w) | 0.59 | 2.09 | 5.19 | 89.57 |
| Heparin + leucine (5% w/w) | 0.61 | 2.16 | 4.77 | 91.18 |
| Heparin + leucine (10% w/w) | 0.58 | 2.04 | 3.93 | 96.6 |
| Heparin + leucine (25% w/w) | 0.63 | 2.34 | 4.85 | 91.15 |
| Heparin + leucine (50% w/w) | 1.05 | 3.03 | 6.62 | 80.03 |

FIGS. 2E to 2H show SEM micrographs of two-fluid nozzle spray dried heparin with 2, 5, 10 and 50% leucine, after secondary drying. When one compares the particles in these Figures to those in FIGS. 2A to 2D, it can be seen that the secondary drying does appear to increase the “collapse” of the particles. Thus, even at, low percentages of FCA, the secondary dried particles have a more wrinkled or shrivelled shape.

TABLE 18
Moisture content of two-fluid nozzle spray
dried particles under standard condition

| | % w/w Moisture before | % w/w Moisture after |
|---|---|---|
| Formulation | secondary drying | secondary drying |
| Heparin + Leucine 5% | 9.57 | 2.18 |

The above discussed experiments and the moisture content values determined by Karl-Fisher methodology set out in Table 18 show that secondary drying significantly reduces the moisture content of heparin particles (by approximately 6.5%). This would imply that the heparin is drying in such a way that there is a hard outer shell holding residual moisture, which is driven off by secondary drying, and further moisture is trapped within a central core. One could infer that the residence time of the particle in the drying chamber is too short, and that the outer shell is being formed rapidly and is too hard to permit moisture to readily escape during the initial spray drying process.

Secondary drying can also be beneficial to the stability of the product, by reducing the moisture content of a powder. It also means that drugs which may be very heat sensitive can be spray dried at lower temperatures to protect them, and then subjected to secondary drying to reduce the moisture further, thereby protecting the drug.

In another embodiment of the invention, the moisture profiling involves increasing the moisture content of the spray dried particles.

Preferably, the moisture is added by exposing the particles to a humid atmosphere. The amount of moisture added can be controlled by varying the humidity and/or the length of time for which the particles are exposed to this humidity.

According to a second aspect of the present invention, compositions are provided comprising spray dried particles comprising a pharmaceutically active agent and a force controlling agent concentrated on the surface of the particles.

In one embodiment, the active agent and FCA were co-spray dried. In another embodiment, the FCA is not an additional, separate material. Rather, the FCA may be the hydrophobic moieties of the active agent arranged on the surface of the particles. Any material included in the particles may be termed an FCA herein if its presence on the surface of the particles has a force controlling effect.

The present invention can be carried out with any pharmaceutically active agent.

The preferred active agents include:

1) steroid drugs such as, for example, alcometasone, beclomethasone, beclomethasone dipropionate, betamethasone, budesonide, clobetasol, deflazacort, diflucortolone, desoxymethasone, dexamethasone, fludrocortisone, flunisolide, fluocinolone, fluometholone, fluticasone, fluticasone proprionate, hydrocortisone, triamcinolone, nandrolone decanoate, neomycin sulphate, rimexolone, methylprednisolone and prednisolone;

2) antibiotic and antibacterial agents such as, for example, metronidazole, sulphadiazine, triclosan, neomycin, amoxicillin, amphotericin, clindamycin, aclarubicin, dactinomycin, nystatin, mupirocin and chlorhexidine;

3) systemically active drugs such as, for example, isosorbide dinitrate, isosorbide mononitrate, apomorphine and nicotine;

4) antihistamines such as, for example, azelastine, chlorpheniramine, astemizole, cetirizine, cinnatizine, desloratadine, loratadine, hydroxyzine, diphenhydramine, fexofenadine, ketotifen, promethazine, trirneprazine and terfenadine;

5) anti-inflammatory agents such as, for example, piroxicam, nedocromil, benzydamine, diclofenac sodium, ketoprofen, ibuprofen, heparinoid, nedocromil, cromoglycate, fasafungine and iodoxamide;

6) anticholinergic agents such as, for example, atropine, benzatropine, biperiden, cyclopentolate, oxybutinin, orphenadine hydrochloride, glycopyrronium, glycopyrrolate, procyclidine, propantheline, propiverine, tiotropium, tropicamide, trospium, ipratropium bromide and oxitroprium bromide;

7) anti-emetics such as, for example, bestahistine, dolasetron, nabilone, prochlorperazine, ondansetron, trifluoperazine, tropisetron, domperidone, hyoscine, cinnarizine, metoclopramide, cyclizine, dimenhydrinate and promethazine;

8) hormonal drugs such as, for example, protirelin, thyroxine, salcotonin, somatropin, tetracosactide, vasopressin or desmopressin;

9) bronchodilators, such as salbutamol, fenoterol and salmeterol;

10) sympathomimetic drugs, such as adrenaline, noradrenaline, dexamfetamine, dipirefin, dobutamine, dopexamine, phenylephrine, isoprenaline, dopamine, pseudoephedrine, tramazoline and xylometazoline;

11) anti-fungal drugs such as, for example, amphotericin, caspofungin, clotrimazole, econazole nitrate, fluconazole, ketoconazole, nystatin, itraconazole, tetbinafine, voriconazole and miconazole;

12) local anaesthetics such as, for example, amethocaine, bupivacaine, hydrocortisone, methylprednisolone, prilocalne, proxymetacaine, ropivacaine, tyrothricin, benzocaine and lignocaine;

13) opiates, preferably for pain management, such as, for example, buprenorphine, dextromoramide, diamorphine, codeine phosphate, dextropropoxyphene, dihydrocodeine, papaveretum, pholcodeine, loperamide, fentanyl, methadone, morphine, oxycodone, phenazocine, pethidine and combinations thereof with an anti-emetic;

14) analgesics and drugs for treating migraine such as clonidine, codine, coproxamol, dextropropoxypene, ergotamine, sumatriptan, tramadol and non-steroidal anti-inflammatory drugs;

15) narcotic agonists and opiate antidotes such as naloxone, and pentazocine;

16) phosphodiesterase type 5 inhibitors, such as sildenafil; and

17) pharmaceutically acceptable salts of any of the foregoing.

A plurality of active agents can be employed in the practice of the present invention.

In preferred embodiments, the active agent is heparin, apomorphine, glycopyrrolate, clomipramine or clobozam.

In view of the increased FPF and FPD obtained, especially when co-spray drying an active agent with an FCA, it may be possible to do away with the large carrier particles in a dry powder comprising an active agent which has been co-spray dried with a force control agent. However, it may still be desirable to include carrier particles, especially where the active agent is to be administered in small amounts, as the bulk of the larger carrier particles will help to ensure that an accurate dose is dispensed.

Preferably, the active agent is a small molecule or the active agent is a carbohydrate, as opposed to a macromolecule. Preferably, the active agent is not a protein or polypeptide, and more preferably, the active agent is not insulin. In the case of proteins and in particular insulin, there is little or no benefit to be derived from the use of a force control agent in a dry powder formulation for administration by inhalation. The reason for this is that in the case of these active agents, the active agent itself acts as a force control agent and the cohesive forces of particles of these active agents are already only weak.

The following is a list of proteins which may be used as the active agent in the compositions and processes according to the present invention. Calcitonin, erythropoetin (EPO), Factor IX, granulocyte colony stimulating factor (G-CSF), granulocyte macrophage colony stimulating factor (GM-CSF), growth hormone, heparin and low molecular weight heparin, insulin type I, interferon alpha, interferon beta, interferon gamma, interleukin-2, luteinizing hormone releasing hormone (LHRH), somatostatin analog, vasopressin analog, follicle stimulating hormone (FSH), amylin, ciliary neurotrophic factor, growth hormone releasing factor (GRF), insulin-like growth factor, insulinotropin, interleukin-1 receptor antagonist, interleukin-3, interleukin-4, Interleukin-6, macrophage colony stimulating factor (M-CSF), nerve growth factor, parathyroid hormone, somatostatin analog, thymosin alpha 1, IIb/IIIa inhibitor, alpha antitrypsin, anti-RSV antibody, cystic fibrosis transmembrane regulator (CFTR) gene, deoxyribonuclease (DNase), bactericidal/permeability increasing protein (BPI), anti-CMV antibody and interleukin-1 receptor.

As discussed above, where the active agent being spray dried includes hydrophobic moieties itself, it is possible to spray dry the active agent without an FCA.

The active agent, preferably, exhibits at least 20, 25, 30, and, more preferably, 40% bioavailability when administered via the lung in the absence of a penetration enhancer. Tests suitable for determining bioavailabihty are well known to those skilled in the art and an example is described in WO 95/00127. Agents that exhibit bio-availability of less than 20%, such as a majority of macromolecules, are insufficiently rapidly cleared from the deep lung and, as a result, accumulate to an unacceptable extent if administered to this location on a long term basis. It is thought that the bioavailability of the active agent may be improved by delivering the active agent to the lung in particles with a size of less than 2 μm, less than 1.5 μm or less than 1 μm. Thus, the spray dried particles of the present invention, which tend to have a particle size of between 0.5 and 5 μm will exhibit excellent bio-availability compared to that of the particles produced by conventional spray drying processes.

In one embodiment of the present invention, the FCAs used are film-forming agents, fatty acids and their derivatives, lipids and lipid-like materials, and surfactants, especially solid surfactants.

Advantageously, the FCA includes one or more compounds selected from amino acids and derivatives thereof, and peptides and derivatives thereof. Amino acids, peptides and derivatives of peptides are physiologically acceptable and give acceptable release of the active particles on inhalation.

It is particularly advantageous for the FCA to comprise an amino acid, and preferably the FCA is a hydrophobic amino acid. The FCA may comprise one or more of any of the following amino acids: leucine, isoleucine, lysine, cysteine, valine, methionine, and phenylalanine. The amino acids leucine, preferably l-leucine, isoleucine, lysine and cysteine have been shown to be particularly effective.

The FCA may be a salt or a derivative of an amino acid, for example aspartame or acesulfame K. Preferably, the FCA consists substantially of an amino acid, more preferably of leucine, advantageously l-leucine. The d- and dl-forms may also be used. As indicated above, l-leucine has been found to give particularly efficient dispersal of the active particles on inhalation.

In another embodiment of the invention, the FCA is not an amino acid. Alternatively, the FCA may not be glycine or alanine.

The FCA may comprise a metal stearate, or a derivative thereof, for example, sodium stearyl fumarate or sodium stearyl lactylate. Advantageously, the FCA comprises a metal stearate. For example, zinc stearate, magnesium stearate, calcium stearate, sodium stearate or lithium stearate.

The FCA may include or consist of one or more surface active materials, in particular materials that are surface active in the solid state. These may be water soluble or able to form a suspension in water, for example lecithin, in particular soya lecithin, or substantially water insoluble, for example solid state fatty acids such as oleic acid, lauric acid, palmitic acid, stearic acid, erucic acid, behenic acid, or derivatives (such as esters and salts) thereof, such as glyceryl behenate. Specific examples of such materials are phosphatidylcholines, phosphatidylethanolamines, phosphatidylglycerols, phosphatidylinositol and other examples of natural and synthetic lung surfactants; lauric acid and its salts, for example, sodium lauryl sulphate, magnesium lauryl sulphate; triglycerides such as Dynsan 118 and Cutina HR; and sugar esters in general. Alternatively, the FCA may be cholesterol or natural cell membrane materials, including pollen or spore cell wall components such as sporo-pollenins.

Other possible FCAs include sodium benzoate, hydrogenated oils which are solid at room temperature. In some embodiments, a plurality of different FCAs can be used.

Alternative FCAs which may be co-spray dried with the active agent include phospholipids and lecithins. However, where the active agent is insoluble in organic solvents, whilst the FCA is insoluble in an aqueous phase, or vice versa, in order to co-spray dry these incompatible materials, one must use a technique such as hydrophobic ion pairing or preferably co-solvents, i.e., a mixture of aqueous and organic solvents.

It is important to note that the particles produced by co-spray drying an active agent and an FCA will comprise both the active agent and the FCA and so the FCA will actually be administered to the lower respiratory tract or deep lung upon inhalation of the dry powder composition. This is in contrast to the additive material used in the prior art, which often was not administered to the deep lung, for example because it remains attached to the large carrier particles.

Thus, it is important that the selected FCA does not have a detrimental effect when administered to the lower respiratory tract or deep lung. Amino acids such as leucine, lysine and cysteine are all harmless in this regard, as are other FCAs such as phospholipids, when present in small quantities.

The above discussion and experiments focussed on spray drying with alternative droplet forming means. However, it should be noted that further changes to the apparatus may be made to ensure that the particles collected at the end of the spray drying process have the optimum properties.

For example, the nature of the drying chamber may be changed, to get better drying and/or other advantages. Thus, in one embodiment of the invention, a spray drying apparatus comprising a drying chamber with heated walls may be used. Such drying chambers are known and they have the advantage that the hot walls discourage deposition of the spray dried material on them. However, the heated walls create a temperature gradient within the drying chamber, where the air in the outer area of the chamber is hotter than that in the centre of the chamber. This uneven temperature can cause problems because particles which pass through different parts of the drying chamber will have slightly different properties as they may well dry to differing extents and at varying rates.

In an alternative embodiment, the spray drying apparatus comprises a radiative heat source in the drying chamber. Such heat sources are not currently used in spray drying. This type of heat source has the advantage that it does not waste energy heating the air in the drying chamber. Rather, only the droplets/particles are heated as they pass through the chamber. This type of heating is more even, avoiding the temperature gradients mentioned above in connection with drying chambers with heated walls. This also allows the particles to dry from inside the droplets thus reducing or avoiding crust forming.

In yet another embodiment of the present invention, the spray dried particles are collected using an upstream vertical drying column. These columns are already known in spray drying devices and they collect the spray dried particles by carrying the particles up a vertical column using an air flow, rather than simply relying on gravity to collect the particles in a collection chamber. The advantage of using such a vertical drying column to collect the spray dried particles is that it allows for aerodynamic classification of the particles. Fine particles tend to be carried well by the air flow, whilst larger particles are not. Therefore, the vertical drying column may separate these larger particles.

Finally, it should be noted that most spray dried powders tend to be amorphous. However, the methods according to the present invention allow amorphous, crystalline powders or any variant (i.e. partially crystalline, nano-crystalline, liquid crystalline phases etc.) to be produced.

What is more, the prior art indicates that amorphous powders are less preferred as they have higher surface energy and higher cohesion. However, where an additive is used to coat an amorphous particle, the nature of the particle beneath that coating no longer affects cohesion, dispersion and other powder properties important in DPIs. Thus, amorphous powders produced according to the methods of the present invention will be suitable for inhalation and can exhibit excellent performance. Consequently, the present invention avoids the previously known problems associated with amorphous powders produced by spray drying, especially where small molecules are being spray dried.

The surface of particles according to the present invention may have some structure, provided by the FCA, as they may exist in lamellar layers, such as those which are common to surfactant types of materials, i.e. have a liquid crystalline structure.

1. A method of making a dry powder composition for pulmonary inhalation, the method comprising spray drying a pharmaceutically active agent in a spray dryer to produce active particles, wherein the step of spray drying further includes producing droplets moving at a controlled velocity.

2. A method as claimed in claim 1, wherein the velocity of droplets at 5 mm from their point of generation is less than 20 m/s.

3. A method as claimed in claim 1, wherein the spray drier comprises an ultrasonic nebuliser.

4. A method as claimed in claim 3, wherein the output of each single nebuliser unit is greater than 5 cc/min.

5. A method as claimed in claim 4, wherein the output of each single nebuliser unit is greater than 10 cc/min.

6. A method as claimed in claim 1, wherein 90% of the resulting dried particles have a size of less than 5 μm, as measured by laser diffraction.

7. A method as claimed in claim 6, wherein 90% of the resulting dried particles have a size of less than 2.51 μm, as measured by laser diffraction.

8. A method as claimed in claim 1, wherein the step of spray drying comprises co-spray drying the active agent with a force control agent.

9. A method as claimed in claim 8, wherein the force control agent is selected from the group consisting of an amino acid, a phospholipid and a metal stearate.

10. A method as claimed in claim 9, wherein the force control agent is selected from the group consisting of leucine, lysine cysteine and combinations thereof.

11. A method as claimed in claim 8, wherein a blend of active agent and force control agent is spray dried, and the blend is a solution.

12. A method as claimed in claim 8, wherein a blend of active agent and force control agent is spray dried, and the blend is a suspension.

13. A method as claimed in claim 11, wherein the active agent and force control agent are spray dried from an aqueous solution or suspension.

14. A method as claimed in claim 1, wherein the step of spray drying comprises co-spray drying the active agent with a force control agent to produce dry particles comprising up to 20% w/w force control agent.

15. A method as claimed in claim 1, wherein the method further comprises adjusting the moisture content of the spray dried particles.

16. A dry powder composition for pulmonary inhalation, wherein the composition is spray dried and comprises particles of a pharmaceutically active material having a force control agent concentrated on the surfaces of the particles.

17. A composition as claimed in claim 16, wherein the composition comprises no more than 20% w/w of an additive which acts as a force control agent.

18. A composition as claimed in claim 16, wherein at least 90% of the particles in the composition have a size of less than 5 μm, as measured by laser diffraction.

19. A composition as claimed in claim 16, wherein the composition has a fine particle fraction of at least 40%.

20. A composition as claimed in claim 16, wherein the composition has a density greater than 0.1 g/cc.

21. A composition as claimed in claim 16, wherein the particles are prepared using a method as claimed in claim 1.

22. A composition as claimed in claim 16, wherein the composition has a fine particle fraction of at least 50%.

23. A composition as claimed in claim 16, wherein the composition has a fine particle fraction of at least 60%.

24. A composition as claimed in claim 16, wherein the composition has a fine particle fraction of at least 70%.
